(19) United States
(12) Patent Application Publication
(10) Pub. No.: US 2026/0152522 A1
(43) Pub. Date: Jun. 4, 2026

(54) DNA-Compatible Wittig and Wittig-Related Olefination of on-DNA Peptidyl-Ylides and Beta-Keto Phosphonates for Development of on-DNA Peptidomimetics through Diversity-Oriented Synthesis (DOS)

(71) Applicant: The Rockefeller University, New York, NY (US)

(72) Inventors: Marc Flajolet, New York, NY (US); Yashoda Sunkari, New York, NY (US)

(21) Appl. No.: 19/126,374

(22) PCT Filed: Nov. 1, 2023
(86) PCT No.: PCT/US2023/078334

§ 371 Date: May 1, 2025

Related U.S. Application Data
(60) Provisional application No. 63/381,865, filed on Nov. 1, 2022.

(51) Int. Cl. C07H 21/04; C07H 1/00; C40B 40/10; C40B 50/08; C40B 70/00

(52) U.S. Cl. CPC C07H 21/04; C07H 1/00; C40B 40/10; C40B 50/08; C40B 70/00

(57) ABSTRACT

The present invention provides methods of generating diverse chemical structures on DNA through Wittig olefination of novel on-DNA phosphorane ylides and Homer-Wadsworth-Emmons reaction of on-DNA β-keto phosphonates. The methods of this invention provide access to DNA-encode libraries (DELs) of diverse peptides, peptidomimetics, chalcone-based molecules, and the like.

### CROSS-REFERENCE TO RELATED APPLICATIONS

This application claims priority under 35 U.S.C. § 119(e) to U.S. Provisional Application No. 63/381,865, filed Nov. 1, 2022, the contents of which are incorporated herein by reference in their entirety.

### STATEMENT REGARDING FEDERALLY SPONSORED RESEARCH OR DEVELOPMENT

This invention was made with government support under W81XHW-17-1-0495 awarded by USA MED RESEARCH ACQ ACTIVITY (USAMRAA). The government has certain rights in the invention.

### BACKGROUND OF THE INVENTION

Structurally, peptides display well-defined secondary structures with complex and greater chirality than smaller molecules, increasing their potency and selectivity compared to small molecular weight molecules (Fosgerau, K., et al., 2015, Drug Discovery Today, 20(1):122-128). The intrinsic properties of natural peptides, such as high susceptibility to proteolysis, are synonym of low bioavailability and rapid clearance, therefore restricting their therapeutic use (Qvit, N., et al., 2017, Drug Discovery Today, 22(2):454-462). Peptidomimetics are modified peptides (including non-natural amino acids) that have been designed to display better pharmacological properties to increase their therapeutic value (Veber, D. F., et al., 1985, Trends in Neurosciences, 8:392-396; Neffe, A. T., et al., 2007, Journal of Medicinal Chemistry. 50(15):3482-3488; Rafi, S. B., et al., 2012, Journal of Medicinal Chemistry, 55(7):3163-3169; Fransson, R., et al., 2013, Journal of Medicinal Chemistry, 56(12):4953-4965; Lenci, A., et al., 2020, Chemical Society Reviews, 49:3262-3277; Muttenthaler, G. E., et al., 2021, Nature Reviews Drug Discovery, 20:309-325). Peptidomimetics might be especially relevant for target mechanisms involving large and flat interfaces including complex hydrophobic and polar interactions such as protein-protein interactions (Stone, T. A., et al., 2018, Bioorganic and Medicinal Chemistry, 26(10):2700-2707). Importantly, very short peptidomimetics retaining the desired properties (e.g., increased selectivity) can act as small molecular weight molecules and they represent novel opportunities to target conventional biological mechanisms such as enzymatic modulation (Makovitzki. A., et al., 2006, Proceedings of the National Academy of Sciences USA, 103(43):15997-16002; Chih, Y. H., et al., 2015. Antimicrobial Agents and Chemotherapy. 59(8):5052-5056; Das. R., et al., 2020, ACS Applied Bio Materials. 3(9):5474-5499).

Numerous peptidomimetic strategies have been developed to reduce conformational flexibility while increasing their binding affinities (Qvit, N., et al., 2017, Drug Discovery Today, 22(2):454-462; Fair. R. J., et al., 2021, Bioorganic and Medicinal Chemistry Letters, 51:128339; Wang. L., et al., 2022, Signal Transduction and Targeted Therapy, 7:48). Novel peptidomimetic compounds have been synthesized through three main routes: 1) local modifications (e.g., peptide backbone isosteres, side-chain modifications and substitution of dipeptide isosteres); 2) generation of novel secondary structure mimetics (e.g., helices, turns, hairpins and sheets); and 3) global restriction by cyclization of linear peptide analogs (e.g., macrocycles).

Side-chain modifications correspond to local modifications of the peptide by incorporating non-natural amino acids harboring appendages. Amino acids presenting side chain functional groups are preferred precursors to be converted into non-natural amino acids by functional group transformations. Various modifications have been developed to modify charged amino acids, for example to reduce or enhance their acidity/basicity, or to substitute aromatic sidechains and heterocycles to generate novel active molecules.

Incorporation of phosphorus into amino acids has been reported to generate phosphonium ylides or β-keto phosphonates, and further transformations lead to a diverse set of α,β-unsaturated molecules (Arribat, M., et al., 2020. RSC Advances, 10:6678-6724).

DNA-encoded library (DEL) technology was initially conceptualized by late Drs. Brenmer and Lemer (Brenner, S., et al., 1992, Proceedings of the National Academy of Sciences, 89(12):5381-5383), and has been considerably developed over the last decade advancing on-DNA compatible chemical reactions, selection methods and high-throughput sequencing. DEL construction is based on large scale combinatorial chemistry obtained by alternating split and pool steps that involve organic synthesis and DNA barcoding (Fair, R. J., et al., 2021, Bioorganic and Medicinal Chemistry Letters, 51:128339; Huang, Y., et al., 2022, Nature Chemistry, 14(2):129-140; Sunkari, Y. K., et al., 2022, Trends in Pharmacological Sciences, 43:4-15; Shi. Y., et al., 2021, RSC Advances, 11:2359-2376). Affinity selection of unprecedented library sizes is now accessible at a miniaturized scale and for a significantly lower cost. A library containing molecular entities that are structurally highly diversified increases the success rate of any drug discovery effort. Due to the fragile nature of DNA, the chemical reaction toolbox to generate DNA-encoded libraries is rather limited as compared to traditional medicinal chemistry synthesis. Despite the difficulties associated with DNA integrity, a number of classical organic reactions have been applied for designing on-DNA compatible reactions (Nielsen, J., et al., 1993, Journal of the American Chemical Society, 115(21):9812-9813: Gartner, Z. J., et al., 2002, Angewandte Chemie International Edition, 41(10):1796-1800; Clark. M. A., et al., 2009: Nature Chemical Biology, 5:647-654: Ding, Y., et al., 2015, ACS Combinatorial Science, 17(1):1-4; Litovchick, A., 2015, Scientific Reports, 5:10916; Li, Y. Z., et al., 2016, ACS Combinatorial Science, 18(8):438-443; Du, H. C., et al., 2017, Bioconjugate Chemistry, 28(10):2575-2580; Lu, X. J., et al., 2017, MedChemComm, 8(8):1614-1617; Skopic, M. K., et al., 2017, Chemical Science, 8:3356-3361; Kolmel, D. K., et al., 2018, ChemMedChem, 13(20):2159-2165; Li, J. Y., et al., 2018, Bioconjugate Chemistry, 29(11):3841-3846; Shu, K., et al., 2018, ACS Combinatorial Science, 20(5):277-281; Wang, J., et al., Proceedings of the National Academy of Sciences USA, 115(28):E6404-E6410; Wang, X., et al., 2018, Organic Letters. 20(16):4764-4768; Zhu, Z., et al., 2018. ACS Chemical Biology. 13(1):53-59; Flood, D. T., et al., 2019, Journal of the American Chemical Society, 141(25):9998-10006; Gerry, C. J., et al., Journal of the American Chemical Society, 141(26):10225-10235; Gironda-Martinez, A., et al., 2019, Organic Letters, 21(23):9555-9558; Kolmel, D. K.. 2019, ACS Combinatorial Science, 21(8):588-597; Kunig, V. B. K., et al., 2019, Organic Letters, 21(18):7238-7243; Li, J. Y., et al., 2019, Bioconjugate Chemistry, 30(8):2209-2215; Phelan, J. P., et al., Journal of the American Chemical Society, 141(8):3723-3732; Wang, X., et al, 2019, Organic Letters, 21(3):719-723; Xu, H. T., et al., 2019, Advanced Science, 6:1901551; Chen, Y. C., et al., 2020, Bioconjugate Chemistry, 31(3):770-780; Fan, Z., et al., 2020, Chemical Science, 31(25):12282-12288; Fitzgerald. P. R., et al., 2020, Chemical Reviews, 121(12):7155-7177; Flood, D. T., et al., 2020, Angewandte Chemie International Edition. 59(19):7377-7383; Kolmel, D. K., et al., 2020, Biochemical and Biophysical Research Communications, 533(2):201-208; Shan, J., et al., 2021, Bioorganic & Medicinal Chemistry, 42:116234; Yang, P., et al., 2021, Chemical Science, 12(16):5804-5810; Adamik, R., 2022, Chemistry, 28(20):e202103967; Eom. S., et al., 2022, Organic Letters. 24(27):4881-4885; Kolusu, S. R. N., et al., 2022, Chemical Science, 13(23):6982-6989; Krumb, M., et al., 2022, Chemical Science, 13(4):1023-1029; Siripuram V. K., 2022, Frontiers in Chemistry, 10:894603; Stanway-Gordon, H. A., et al., 2022, Angewandte Chemie International Edition, 61(3):e202111927; Xu, H. T., et al., Advanced Science, 9(26):2202790; Yen-Pon, E., et al., 2022, Journal of the American Chemical Society, 144(27):12184-12191; Zhong, S., et al., 2022, Organic Letters, 24(4):1022-1026; Satz, A. L., et al., 2015, Bioconjugate Chemistry, 26(8):1623-1632). To further increase the chemical diversity of DEL, new DNA-compatible reactions are needed. The possibility to couple on-DNA combinatorial chemistry to diversity-oriented synthesis (DOS) will further expand the chemical diversity of DELs (Wu, R. F., et al., 2020, Chemistry—an Asian Journal, 15(23):4033-4037; Liu. S., et al., 2021. Organic Letters, 23(3):908-913).

Wittig reaction compatible with a DNA-templated library has been initially reported to generate an α,β-unsaturated amide by Liu and co-workers (Gartner, Z. J., et al., 2002, Angewandte Chemie International Edition, 41(10):1796-1800; Gartner, Z. J., 2002, Journal of the American Chemical Society, 124(35):10304-10306; Gartner, Z. J., et al., 2004, Science, 305(5690):1601-1605).

However, DNA-templated reactions come with practical limitations in the context of split-and-pool strategies as each building block involved is required to harbor two functional groups, one for the reaction and one to conjugate the DNA fragment.

Recently, Yu-Long An et al. reported the use of DNA-compatible Wittig reaction suitable to generate α, β-unsaturated amide moiety in the context of split-and-pool strategy (An, Y. L., et al., 2020, Organic Letters, 22(10):3931-3935). In that case, the DNA-compatible Wittig reaction used to generate α,β-unsaturated ketones was restricted to α-bromo ketones containing building blocks which are not readily available and rare commercially. Additionally, creating an unsaturated ketone from β-keto phosphonate has not been explored on DNA, which are in general more stable than the Wittig ylides.

Thus, there is a need in the art for a method of generating α,β-unsaturated ketones on-DNA that is applicable to diverse building blocks. The present invention satisfies the need in the art.

### SUMMARY

The present invention relates to methods for synthesizing compounds for DNA encoded libraries (DELs) on DNA, compounds useful for preparing the DELs, and the compounds prepared through the methods. The invention is based, in part, on the development of phosphorane ylides which can be prepared on DNA in DNA-stable conditions. The invention is also based, in part, on the development of phosphorane ylides which can react with a wide range of compounds under conditions amenable to DNA.

In one embodiment, the method of preparing compounds for DELs comprises (a) preparing a DNA barcode; (b) preparing a phosphonium salt; and (c) conjugating the phosphonium salt to a DNA molecule to yield an on-DNA phosphonium ylide.

In some embodiments, the method further comprises step (d) deprotecting an on-DNA phosphonium ylide. In some embodiments, the method further comprises step (e) conjugating a carboxylic acid, including but not limited to an amino acid, to a deprotected on-DNA phosphonium ylide. In one embodiment, the method further comprises one or more steps (f) repeating steps (d) and (e) with carboxylic acids of interest being added to a deprotected amine of a previously added carboxylic acid to prepare a peptide or peptidomimetic.

In one embodiment, the method further comprises step (g) reacting a phosphorane ylide with an aldehyde to yield the corresponding α,β-unsaturated ketone. In some embodiments, the method further comprises step (h) reacting a α,β-unsaturated ketone with a nucleophilic compound.

In some embodiments, the present disclosure provides compounds for preparing on-DNA-phosphorane ylide on-DNA-phosphorane ylides. In various embodiments, the compound for preparing an on-DNA-phosphorane ylide is a compound of Formula (II):

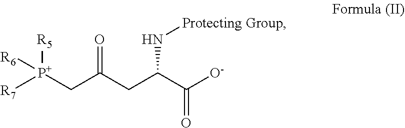

wherein each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, the invention provides compounds of Formula (III) for generating compounds to add to a DEL, wherein Formula (III) is:

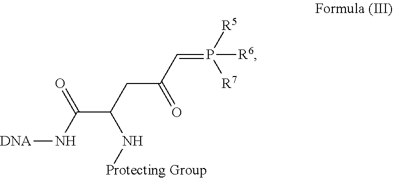

wherein each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, the invention provides compounds to add to a DEL of Formula (XII):

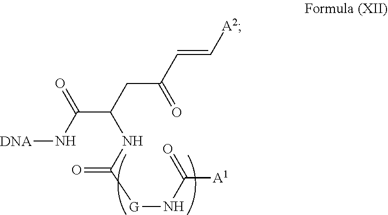

wherein each instance of G is independently of the formula

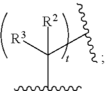

each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R², or R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a synthetic scheme for on-DNA aspartic acid-derived phosphorane ylides. Reaction conditions: i) BTFFH, DCM, DIPEA, room temperature, overnight; ii) 9:1 TFA/DCM, room temperature, 4 hours; iii) MPOS, 0.5 M NaCl in DMSO, EDC, HOAt, DIPEA, pH 8.0, 20° C. 4 hours.

FIG. 2, comprising FIGS. 2A and 2B, depicts representative LC-MS profiles of on-DNA aspartic acid-derived phosphorane ylides. FIG. 2A depicts a representative total ion and UV chromatogram and deconvoluted mass spectrum of compound 4a. FIG. 2B depicts a representative total ion and UV chromatogram and deconvoluted mass spectrum of compound 4b.

FIG. 3 depicts a synthetic scheme for deprotection of the Wittig ylide in the presence of piperidine. Reaction conditions: i) 10% piperidine in 1120, 20° C.; ii) MOPS, 0.5 M NaCl in DMSO, benzoic acid, EDC, HOAt, DIPEA, pH 8.0, 20° C., 4 hours.

FIG. 4, comprising FIGS. 4A and 4B, depicts representative LC-MS profiles of on-DNA aspartic acid-derived phosphorate ylides after exposure to 10% piperidine. FIG. 4A depicts a representative total ion and UV chromatogram and deconvoluted mass spectrum, of compound 5a after piperidine treatment for 3 or 24 hours. FIG. 4B depicts a representative total ion and UV chromatogram and deconvoluted mass spectrum of compound 5b after piperidine treatment for 3 or 24 hours.

FIG. 5 depicts a synthetic scheme for Wittig reaction condition optimization. Optimization reaction results are summarized in Table 1.

FIG. 6 depicts a synthetic scheme for synthesizing α,β-unsaturated ketones on-DNA from aspartic acid-derived triphenylphosphoranylidene 6 and a representative set of compounds demonstrating substrate scope. Reaction conditions: Aldehyde (1000 equiv.), DIPEA, (1000 equiv.), H₂O:CH₃CN (3:1), 50° C., 12 hours; ^{a}60° C.; ^{b}40° C.

FIG. 7 depicts a synthetic scheme for preparing representative peptide scaffolds having an aspartic acid-derived Wittig ylide. Reaction conditions: i) MOPS, 0.5 M NaCl in DMSO, Fmoc-Phe-OH, EDC, HOAt, DIPEA. pH 8.0, 20° C., 4 hours: ii) 10% piperidine in H₂O, 20° C., 1 hour; iii) MOPS, 0.5 M NaCl in DMSO, benzoic acid, EDC, HOAt, DIPEA. pH 8.0, 20° C. 4 hours; iv) aldehyde, DIPFA, H₂O/CH₃CN, 50° C.; v) MOPS 0.5 M NaCl in DMSO, Fmoc-Leu-OH, EDC. HOAt, DIPEA, pH 8.0, 20° C. 4 hours; vi) MOPS, 0.5 M NaCl in DMSO, Fmoc-Gly-OH, EDC, HOAt, DIPEA, pH 8.0, 20° C., 4 hours.

FIG. 8 depicts representative structures of various peptides synthesized from aspartic acid-derived peptidyl-ylides based on the synthetic scheme of FIG. 7.

FIG. 9 depicts a representative UV chromatogram and mass spectra of compound 13; expected MW: 628; observed MW: 628 and 610.

FIG. 10 depicts a representative UV chromatogram and mass spectrum of compound 14; expected MW. 425; observed MW: 425.

FIG. 11 depicts a synthetic scheme of on-DNA peptidomimetics containing various heterocyclic cores from on-DNA α,β-unsaturated peptidyI ketones through a DOS strategy. Reaction conditions i) NH₂OH·HCl, phosphate buffer, pH 5.5, 60° C., 2 hours; ii) NH₂NH₂·H₂O, borate buffer, pH 9.4, 30° C. 3 hours; iii) guanidine hydrochloride, NaOH, 70° C., 3 hours.

FIG. 12 depicts a synthetic scheme for the synthesis of on-DNA aryl-derived phosphorane ylides and aldehydes representative of the reaction scope. Reaction conditions: aldehyde (1000 equiv.), DIPEA (1000 equiv.), H₂O:CH₃CN (3′1), 50° C. 12 hours; ^{a}60° C.; ^{b}40° C.

FIG. 13 depicts a synthetic scheme for the synthesis of Asp-derived β-keto-phosphonate-based amino acids.

FIG. 14 depicts a synthetic scheme for the synthesis of Asp-derived β-keto-phosphonate tetrapeptides (20) and tripeptides (21). Reaction conditions: (i) MOPS, pH 8.0, 0.5 M NaCl/DMSO, 17, EDC, HOAt, DIPEA, 20° C., 4 h; (ii) 2% Piperidine in H₂O, 20° C., 1 h; (iii) MOPS, pH 8.0, 0.5 M NaCl/DMSO, Fmoc-Ile-OH, EDC, HOAt, DIPEA, 20° C., 4 h; (iv) MOPS, pH 8.0, 0.5 M NaCl/DMSO, Fmoc-Val-OH, EDC. HOAt, DIPEA, 20° C., 4 h; v) MOPS, pH 8.0, 0.5 M NaCl/DMSO, Fmoc-Tvr-OH, EDC, HOAt, DIPEA. 20° C., 4 h; (vi) MOPS, pH 8.0, 0.5 M NaCl/DMSO. Fmoc-Phe-OH, EDC, HOAt, DIPEA, 20° C., 4 h; (vii) MOPS, pH 8.0, 0.5 M NaCl/DMSO, Benzoic acid, EDC, HOAt, DIPEA, 20° C., 4 h.

FIG. 15 depicts a synthetic scheme for optimization of Horner-Wadsworth-Emmons reactions between DNA conjugated tetrapeptide-based-β-keto phosphonate and benzaldehyde. Optimization reaction results are depicted in Table 2.

FIG. 16 depicts representative aldehydes utilized in Horner-Wadsworth-Emmons reactions with β-keto-phosphonate-tetrapeptide. Reaction conditions for H4, H9, H10, H12-H16, and H20-H23: RCHO (1,000 equiv.), KOH (500 equiv.), H₂O:ACN (1:1), 40° C.; ^{a}=25° C. Reaction conditions for H2, H3, H5, H8, and H11: RCHO (1,000 equiv.), K₂CO₃ (500 equiv.), H₂O:ACN (1:1), 60° C. Reaction conditions for H6, H7, and H17-H19: RCHO (500 equiv.), K₂CO₃ (500 equiv.), H₂O:ACN (1:1), 60° C.

FIG. 17 depicts representative aldehydes utilized in Horner-Wadsworth-Emmons reactions with β-keto-phosphonate-tripeptide. Reaction conditions for H24, H27-H30. H37, H38, H40, H41, H45, and H46: RCHO (1,000 equiv.), KOH (500 equiv.), H₂O:ACN (1:1), 40° C.: ^{a}=25° C. Reaction conditions for H25, H26, H31, H32, and H36: RCHO (1,000 equiv.). K₂CO₃ (500 equiv.). H₂O:ACN (1:1), 60° C. Reaction conditions for H33-H35 and H39-H44: RCHO (500 equiv.), K₂CO₃ (500 equiv.), H₂O:ACN (1:1), 60° C.

### DETAILED DESCRIPTION

The present invention relates to methods for synthesizing compounds for DNA encoded libraries (DELs) on natural or synthetic DNA, methods for synthesizing compounds on DNA (natural or synthetic), RNA, or any nucleic acid, compounds useful for preparing DELs, and the compounds prepared through the methods. In one embodiment, the methods of the invention have been developed for producing DELs of peptides and peptidomimetics on DNA, increasing the scope of compounds which may be directly generated on the corresponding DNA tag. The invention is based, in part, on the development of phosphorane-ylides which can be prepared on DNA in suitable conditions. The invention is also based, in part, on the development of phosphorane ylides which can react with a wide range of compounds under conditions amenable to DNA.

In one embodiment, the method of preparing compounds for DELs comprises (a) preparing a barcode; (b) preparing a phosphonium salt; and (c) conjugating the phosphonium salt to a DNA molecule to yield an on-DNA phosphonium ylide.

In some embodiments, the barcode of step (a) is a double stranded DNA sequence 6-500 nucleotides in length with a single overhang of 1-100 nucleotides in length, optionally conjugated to a primary amine by a polyethylene glycol (PEG) linker, wherein the PEG linker is 1, 2, 3, 4, 5, 6, 7, 8, 9, 10, 11, 12, 13, 14, or 15 PEG units in length.

In some embodiments, step (b) yields a compound of Formula (II):

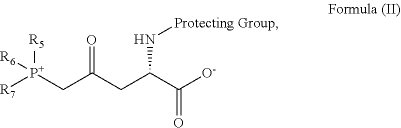

wherein each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, step (c) yields a compound of Formula (III):

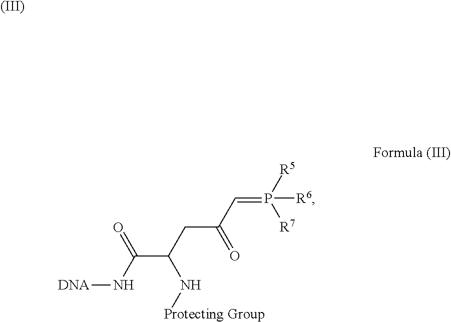

wherein DNA is the barcode of step (a);
each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In one embodiment, the method of preparing compounds for DELs comprises (a) preparing a barcode; (b) preparing a β-keto phosphonate; and (c) conjugating the β-keto phosphonate to a DNA molecule to yield an on-DNA β-keto phosphonate.

In some embodiments, the barcode of step (a) is a double stranded DNA sequence 6-500 nucleotides in length with a single overhang of 1-100 nucleotides in length, optionally conjugated to a primary amine by a polyethylene glycol (PEG) linker, wherein the PEG linker is 1, 2, 3, 4, 5, 6, 7, 8, 9, 10, 11, 12, 13, 14, or 15 PEG units in length.

In some embodiments, the β-keto phosphonate is a compound of Formula (IV):

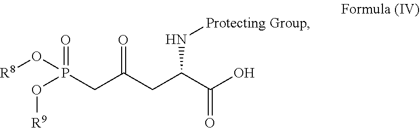

wherein each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, step (c) yields a compound of Formula (V):

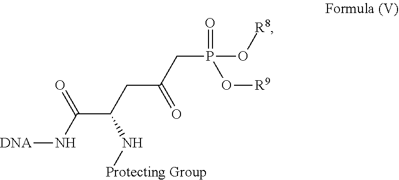

wherein DNA is the barcode of step (a):
wherein each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof, and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, the method further comprises step (d) deprotecting the on-DNA phosphonium ylide or on-DNA β-keto phosphonate. In some embodiments, the method further comprises step (e) conjugating a carboxylic acid, including but not limited to an amino acid, a non-naturally encoded amino acid, a protected amino acid, and a protected non-naturally encoded amino acid, to the deprotected on-DNA phosphonium ylide or on-DNA β-keto phosphonate. In one embodiment, the method further comprises one or more steps (f) repeating steps (d) and (e) with carboxylic acids of interest being added to a deprotected amine of the previously added carboxylic acid to prepare a peptide or peptidomimetic.

In some embodiments, step (d) yields a compound of Formula (VI):

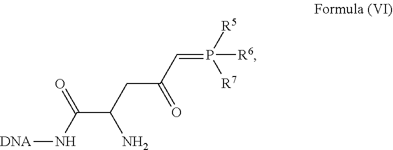

wherein DNA is the barcode of step (a); and
each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In some embodiments, step (d) yields a compound of Formula (VII):

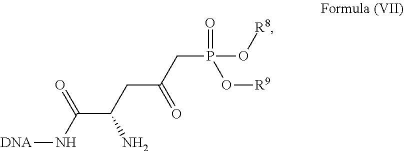

wherein DNA is the barcode of step (a); and
each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In some embodiments, step (e) yields a compound of Formula (VIII):

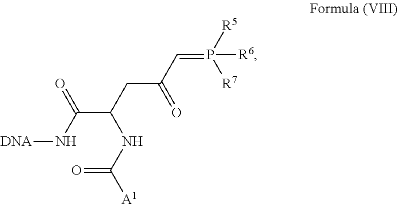

wherein DNA is the barcode of step (a):
A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In some embodiments, step (e) yields a compound of Formula (IX):

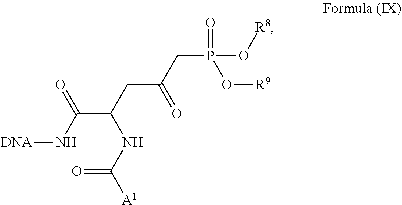

wherein DNA is the barcode of step (a);
A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In some embodiments, the method comprises 1-20 steps (f), which yields a compound of Formula (X):

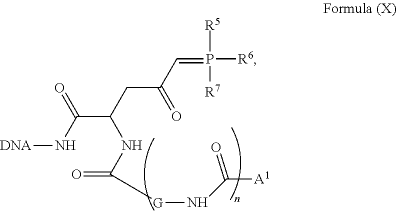

wherein DNA is the barcode of step (a):
each instance of G is independently of the formula

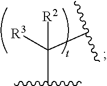

each occurrence of A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
each occurrence of R⁴ is independently selected from the group consisting of H, an optionally substituted C₁-C₁₀ alkyl, and an optionally substituted aryl-(C₁-C₄)alkyl;
each occurrence of R⁵. R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of t is independently an integer of 1-10; and
n is an integer of 1-20.

In some embodiments, the method comprises 1-20 steps (f), which yields a compound of Formula (XI):

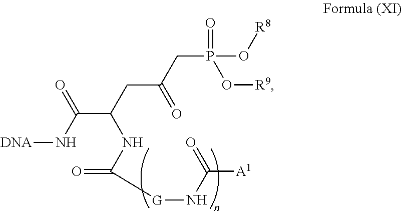

wherein DNA is the barcode of step (a);
each instance of G is independently of the formula

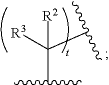

each occurrence of A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
each occurrence of R⁴ is independently selected from the group consisting of H, an optionally substituted C₁-C₁₀ alkyl, and an optionally substituted aryl-(C₁-C₄)alkyl;
each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of t is independently an integer of 1-10; and
n is an integer of 1-20.

In one embodiment, the method further comprises step (g) reacting the phosphorane ylide or β-keto phosphonate with an aldehyde to yield the corresponding α,β-unsaturated ketone. In some embodiments, the method further comprises step (h) reacting the α,β-unsaturated ketone with a nucleophilic compound.

In some embodiments, step (g) yields a compound of Formula (XII):

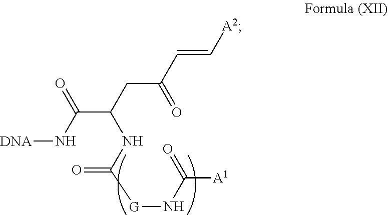

wherein DNA is the DNA barcode of step (a);
each instance of G is independently of the formula

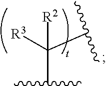

each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R¹, R², and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and
each occurrence of R⁴ is independently selected from the group consisting of H, an optionally substituted C₁-C₁₀ alkyl, and an optionally substituted aryl-(C₁-C₄)alkyl:
each occurrence of t is independently an integer of 1-10; and
n is an integer of 1-20.

In some embodiments, step (h) yields a compound of Formula (XIII):

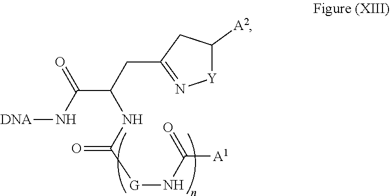

and
wherein DNA is the barcode of step (a):
each instance of G is independently of the formula

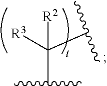

each occurrence of A¹ and A² is independently selected from the group consisting of H, halo, amino, thio, hydroxyl, C₁-C₁₀ alkyl, C₁-C₁₀ alkenyl. C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

In some embodiments, step (h) yields a compound of Formula (XIV):

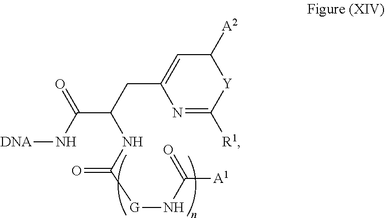

wherein DNA the DNA barcode of step (a):
each instance of G is independently of the formula

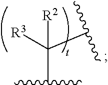

each occurrence of A¹ and A² is independently selected from the group consisting of H, halo, amino, thio, hydroxyl. C₁-C₁₀ alkyl, C₁-C₁₀ alkenyl. C₁-C₁₀ alkynyl, C₁-C₁₀ alkoxy. C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

An “amino terminus modification group” refers to any molecule that can be attached to the amino terminus of a polypeptide. Similarly, a “carboxy terminus modification group” refers to any molecule that can be attached to the carboxy terminus of a polypeptide. Terminus modification groups include but are not limited to various water soluble polymers, peptides or proteins such as serum albumin, or other moieties that increase serum half-life of peptides.

As used herein, “derivatives” are compositions formed from the native compounds either directly, by modification, or by partial substitution. As used herein, “analogs” are compositions that have a structure similar to, but not identical to, the native compound.

As used herein, the terms “amino acid”. “amino acidic monomer”, or “amino acid residue” refer to any of the twenty naturally occurring amino acids including synthetic amino acids with unnatural side chains and including both D and L optical isomers.

In the context of the invention, term “natural amino acid” means any amino acid which is found naturally in vivo in a living being. Natural amino acids therefore include amino acids coded by mRNA incorporated into proteins during translation but also other amino acids found naturally in vivo which are a product or by-product of a metabolic process, such as for example ornithine which is generated by the urea production process by arginase from L-arginine. In the invention, the amino acids used can therefore be natural or not. Namely, natural amino acids generally have the L configuration but also, according to the invention, an amino acid can have the L or D configuration.

A “non-naturally encoded amino acid” refers to an amino acid that is not one of the 20 common amino acids or pyrolysine or selenocysteine. The term “non-naturally encoded amino acid” includes, but is not limited to, amino acids that occur naturally by modification of a naturally encoded amino acid (including but not limited to, the 20 common amino acids or pyrolysine and selenocysteine) but are not themselves incorporated into a growing polypeptide chain by the translation complex, derivatives of amino acids, and non-naturally occurring amino acids. Examples of naturally-occurring amino acids that are not naturally-encoded include, but are not limited to, N-acetylglucosaminyl-L-serine, N-acetylglucosaminyl-L-threonine, and O-phosphotyrosine.

A “barcode”, as used herein, refers to a nucleotide sequence that serves as a means of identification for compound of the present invention. Barcodes of the present invention may comprise at least 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 400, 450, or about 500 nucleotides. In one embodiment, the barcode further comprises a single overhang that is at least 1, 2, 3, 4, 5, 6, 7, 8, 9, 10, 11, 12, 13, 14, 15, 20, 25, 50, 75, or about 100 nucleotides in length. The nucleotide sequence may be a natural or synthetic DNA sequence, a natural or synthetic RNA sequence, or any other sequence of natural or synthetic nucleotides A barcode may consist of the nucleic acid molecule alone or may comprise the nucleic acid and a linker.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. Furthermore, peptides of the invention may include amino acid mimentics, and analogs. Recombinant forms of the peptides can be produced according to standard methods and protocols which are well known to those of skill in the art, including for example, expression of recombinant proteins in prokaryotic and/or eukaryotic cells followed by one or more isolation and purification steps, and/or chemically synthesizing peptides or portions thereof using a peptide sythesizer.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl,” “haloalkyl,” and “homoalkyl.”

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy. —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

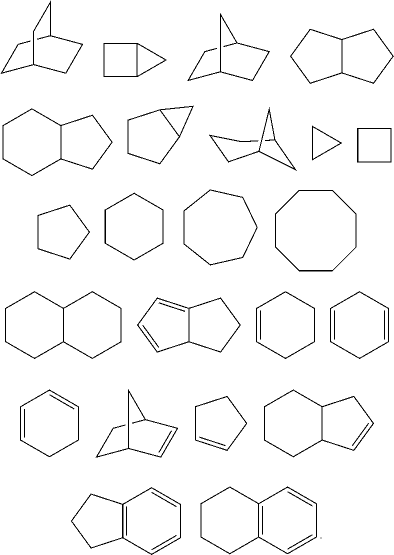

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon-carbon double bond or one carbon-carbon triple bond.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

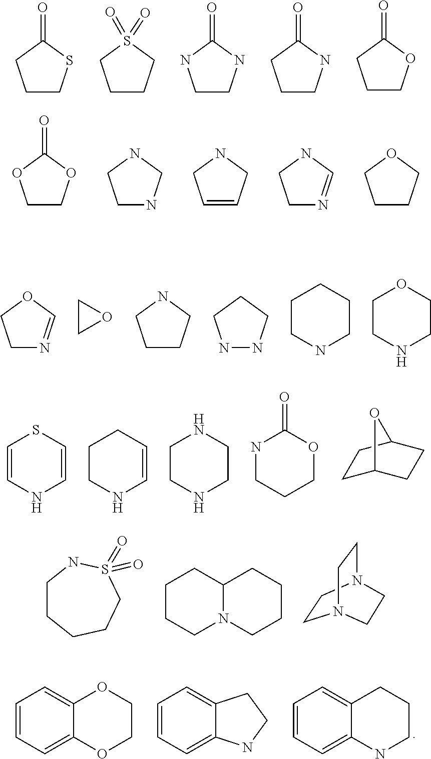

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₄)alkyl” means a functional group wherein a one to three carbon alkylene chain is attached to an aryl group, e.g., —CH₂CH₂-phenyl. The term “substituted aryl-(C₁-C₄)alkyl” means an aryl-(C₁-C₄)alkyl functional group in which the aryl group is substituted. Similarly, the term “heteroaryl-(C₁-C₄)alkyl” means a functional group wherein a one to three carbon alkylene chain is attached to a heteroaryl group, e.g., —CH₂CH₂-pyridyl. The term “substituted heteroaryl-(C₁-C₄)alkyl” means a heteroaryl-(C₁-C₄)alkyl functional group in which the heteroaryl group is substituted.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl). 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-. 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₄)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂ alkyl. —C(═O)NH[substituted or unsubstituted alkyl, or substituted or unsubstituted phenyl], —C(═O)N[H or alkyl]2, —OC(═O)N[substituted or unsubstituted alkyl]2, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl]. —C(OH)[substituted or unsubstituted alkyl]2, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃. —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein but differing from it in respect to certain components or structural makeup, which may have a similar or opposite action metabolically. An analog or derivative can also be a small molecule that differs in structure from the reference molecule but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention provides methods of preparing compounds for DELs, including methods of preparing intermediate compounds. This invention also includes the intermediate compounds and final compounds for addition to or creation of a DEL. This invention includes the use of these methods or compounds produced by these methods, alone or in any combination, to generate a DEL.

### Compounds for Preparation of On-DNA-Phosphorane Ylide On-DNA-Phosphorane Ylides

The present disclosure is based, in part, on the discovery of DNA-compatible Wittig olefination reactions. These reactions are made possible by novel phosphorane ylides which can be prepared on a molecule of DNA. As such, in certain embodiments, this invention provides compounds which are on-DNA-phosphorane ylide on-DNA-phosphorane ylides.

In some embodiments, the present disclosure provides intermediate compounds for the formation of on-DNA phosphorane ylides. In various embodiments, the intermediate compound is of the Formula (I):

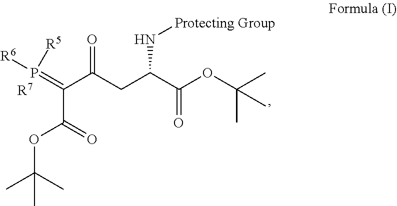

wherein each instance of each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In one embodiment, the compound of Formula (I) is

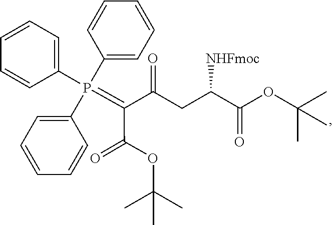

In one embodiment, the intermediate compound for the formation of DNA phosphorane ylides is a compound of Formula (Ib):

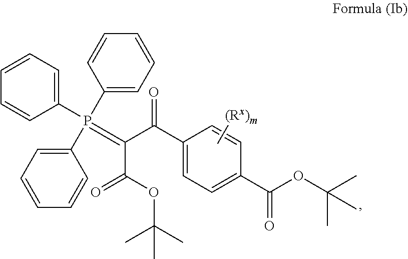

wherein each occurrence of R^{X} is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl; and
m is an integer of 1-4.

In one embodiment, the compound of Formula (Ib) is

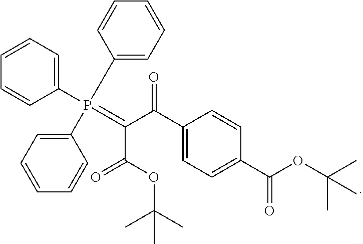 [SMILES: CC(C)(C)OC(=O)C(C(=O)c1ccc(C(=O)OC(C)(C)C)cc1)=P(c1ccccc1)(c1ccccc1)c1ccccc1]

In some embodiments, the present disclosure provides compounds for preparing on-DNA-phosphorane ylide on-DNA-phosphorane ylides. In various embodiments, the compound for preparing an on-DNA-phosphorane ylide is a compound of Formula (II):

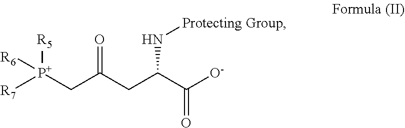

wherein each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In one embodiment, the compound of Formula (II) is

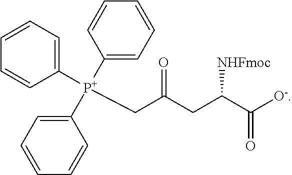

In one embodiment, the compound for preparing a DNA phosphorane ylide is a compound of Formula (IIb):

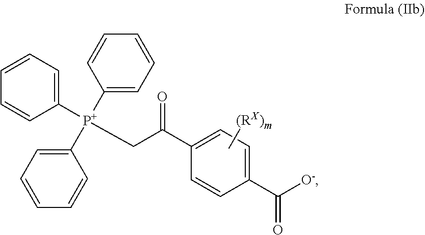

wherein each occurrence of R^{X} is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl; and
m is an integer of 1-4.

In one embodiment, the compound of Formula (IIb) is

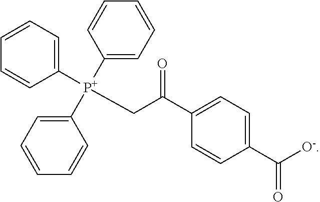

In some embodiments, the present invention provides on-DNA-phosphorane ylide on-DNA-phosphorane ylides. In various embodiments, the on-DNA-phosphorane ylide is a compound of Formula (III):

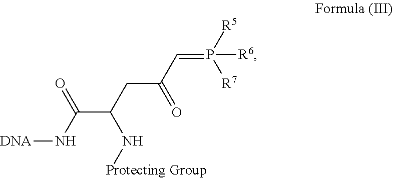

wherein each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, the compound of Formula (III) is a compound of Formula (IIIa):

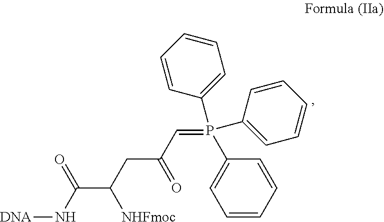

wherein DNA is a nucleic acid barcode.

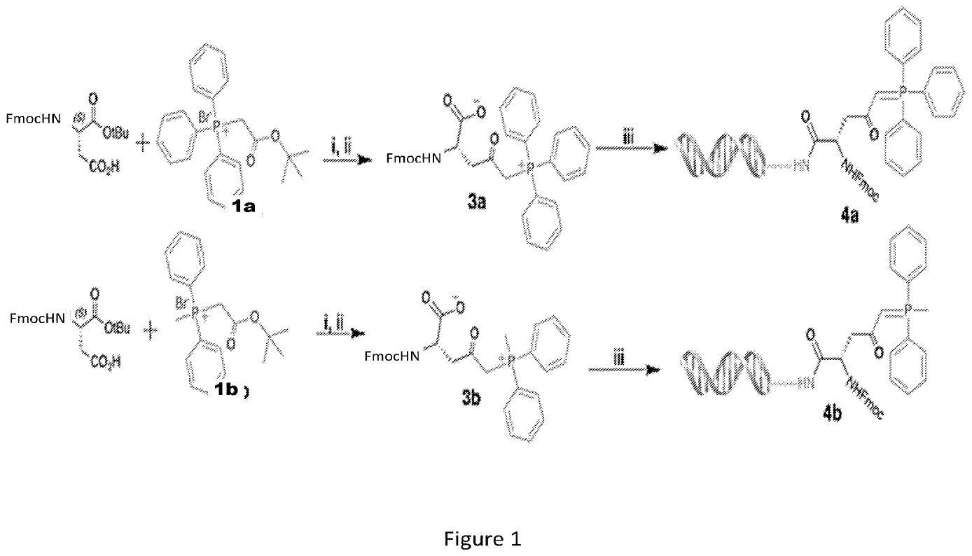

In one embodiment, the compound of Formula (III) has a structure selected from the group consisting of at least one structure as shown in FIG. 1.

In some embodiments, the DNA phosphorane ylide is a compound of Formula (XV):

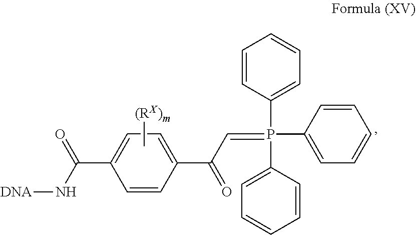

wherein DNA is a nucleic acid barcode;
each occurrence of R^{X} is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl; and
m is an integer of 1-4.

In one embodiment, the compound of Formula (XV) is a compound of Formula (XVa):

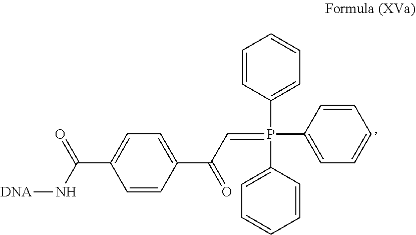

wherein DNA is a nucleic acid barcode.

In some embodiments, the present disclosure provides compounds for preparing on-DNA β-keto phosphonates. In various embodiments, the compound for preparing an on-DNA β-keto phosphonate is a compound of Formula (IV):

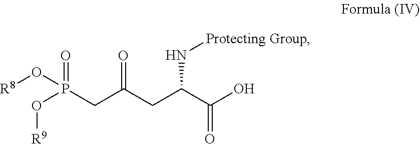

wherein each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof, and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, the compound of Formula (IV) is

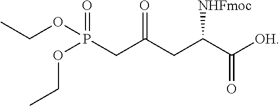

In some embodiments, the β-keto phosphonate is a compound of Formula (IVb):

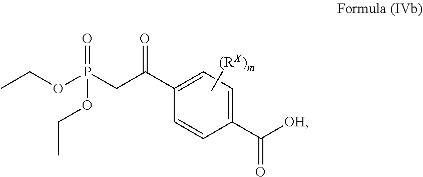

wherein each occurrence of R^{X} is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl; and
m is an integer of 1-4.

In some embodiments, the compound of Formula (IVb) is

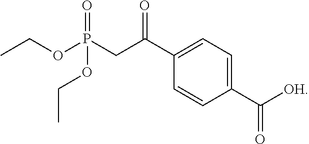 [SMILES: CCOP(=O)(CC(=O)c1ccc(C(=O)O)cc1)OCC]

In some embodiments, the present invention provides on-DNA β-keto phosphonates. In some embodiments, the on-DNA f-keto phosphonate is a compound of Formula (V):

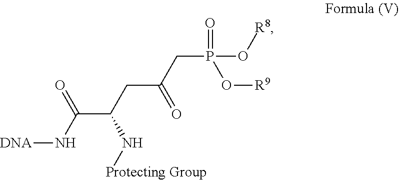

wherein DNA is a nucleic acid barcode;
wherein each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, the compound of Formula (V) is a compound of formula (Va):

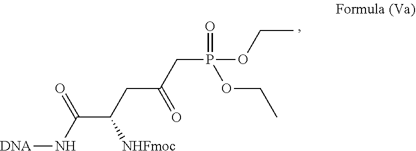

wherein DNA is a nucleic acid barcode.

In some embodiments, the on-DNA β-keto phosphonate is a compound of formula (XVI):

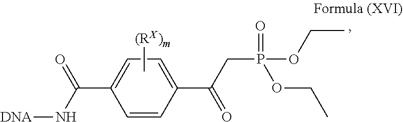

wherein DNA is a nucleic acid barcode;
each occurrence of R^{X} is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl; and
m is an integer of 1-4.

In one embodiment, the compound of formula (XVI) is a compound of formula (XVIa):

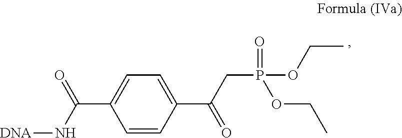

wherein DNA is a nucleic acid barcode.

In some embodiments, the N forming the amide bond of a compound of Formula (III), (IIIa), (V), (Va), (XV), (XVa), (XVI), or (XVIa) is any primary nitrogen in the DNA barcode. In some embodiments, the N forming the amide bond is a primary nitrogen from an unpaired nucleotide on a single-strand overhang in the DNA barcode. In some embodiments, the N forming the amide bond is a primary nitrogen of a linker that is conjugated to the DNA barcode.

Compounds for Entry into DELs

The present invention is based, in part, on the preparation of compounds for entry into a DNA-encoded library (DEL).

In some embodiments, the compound for addition to a DEL is a compound of Formula (VI):

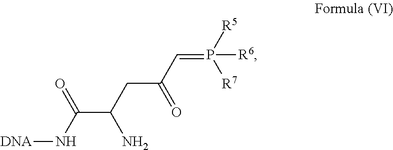

wherein DNA is a nucleic acid barcode; and
each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In one embodiment, the compound of Formula (VI) is a compound of Formula (VIa):

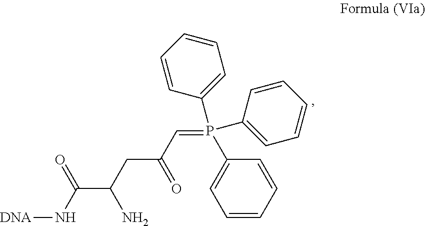

wherein DNA is a nucleic acid barcode.

In some embodiments, the compound for addition into a DEL is a compound of Formula

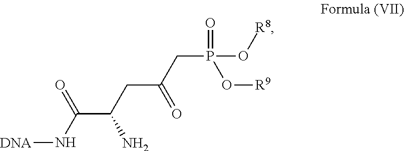

wherein DNA a nucleic acid barcode; and
each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In some embodiments, the compound of Formula (VII) is a compound of Formula (VIIa):

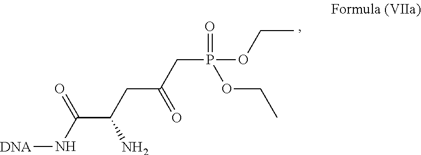

wherein DNA a nucleic acid barcode.

In some embodiments, the compound for addition to a DEL is a compound of Formula (VIII):

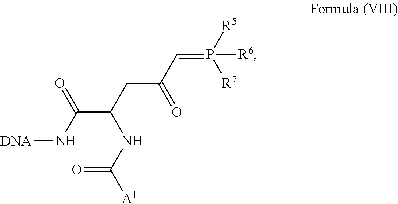

wherein A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In some embodiments, the compound of Formula (VIII) is a compound of Formula (VIIIa):

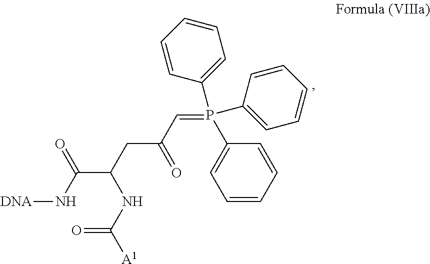

wherein DNA is a nucleic acid barcode; and
wherein A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁O cycloalkyl, C₁-C₁₀ heteroalkyl, C₂-C₁₀ heterocyclyl, aryl, aryl-(C₁-C₄)alkyl, heteroaryl, and any combination thereof.

In some embodiments, the compound for addition to a DEL is a compound of Formula (IX):

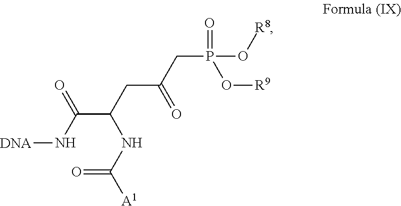

wherein DNA is a nucleic acid barcode:
A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁O cycloalkyl, C₁-C₁₀ heteroalkyl, C₂-C₁₀ heterocyclyl, aryl, aryl-(C₁-C₄)alkyl, heteroaryl, and any combination thereof.

In some embodiments, the compound of Formula (IX) is a compound of Formula (IXa):

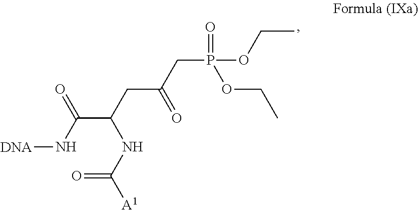

wherein DNA is a nucleic acid barcode;
A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In some embodiments, the compound for addition to a DEL is a compound of Formula

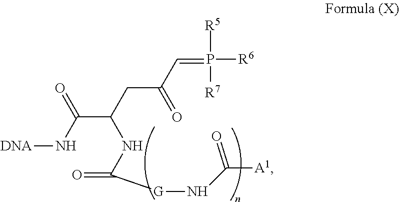

wherein DNA is a nucleic acid barcode:
wherein each instance of G is independently of the formula

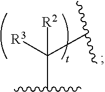

each occurrence of A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴;
each occurrence of R⁴ is independently selected from the group consisting of H, an optionally substituted C₁-C₁₀ alkyl, and an optionally substituted aryl-(C₁-C₄)alkyl;
each occurrence of R⁵, R⁶, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of t is independently an integer of 1-10; and
n is an integer of 1-20.

In one embodiment, the compound of Formula (X) is a compound of Formula (Xa):

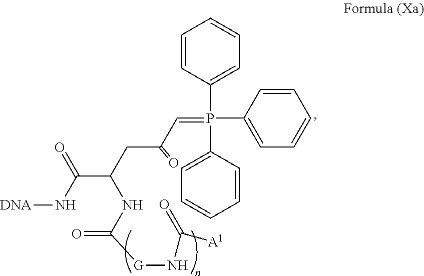

wherein each instance of G is independently of the formula

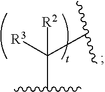

each occurrence of A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
each occurrence of R¹, R², and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

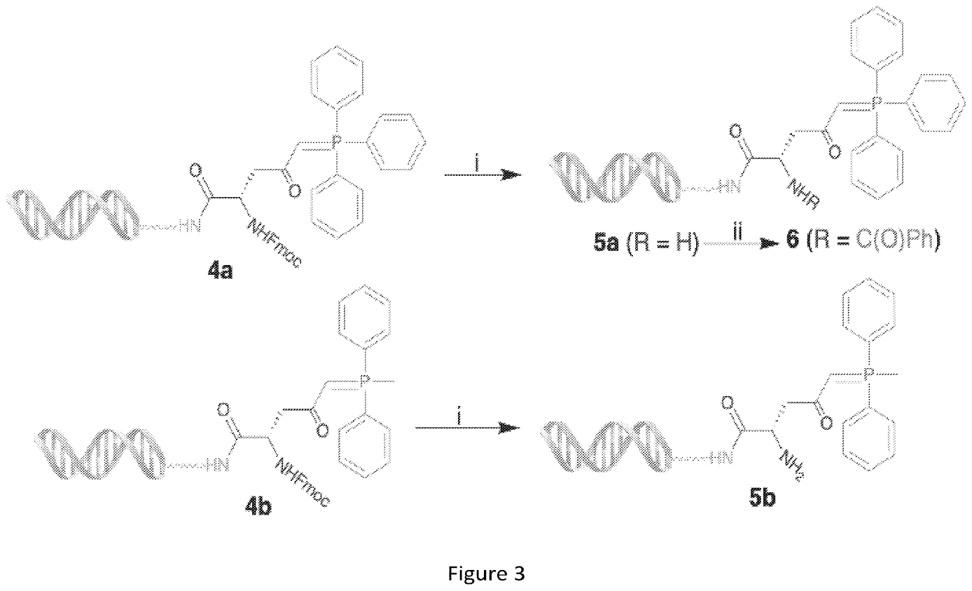

In one embodiment, the compound of Formula (X) has a structure selected from the group consisting of at least one structure as shown in FIG. 3.

In some embodiments, the compound for addition to a DEL is a compound of Formula (XI):

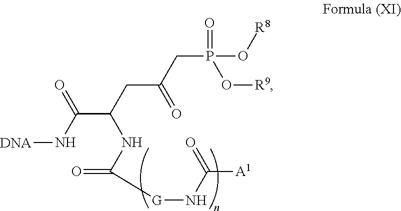

wherein DNA is a nucleic acid barcode;
each instance of G is independently of the formula

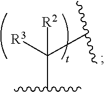

each occurrence of A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
each occurrence of R⁴ is independently selected from the group consisting of H, an optionally substituted C₁-C₁₀ alkyl, and an optionally substituted aryl-(C₁-C₄)alkyl;
each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof:
each occurrence of t is independently an integer of 1-10; and
n is an integer of 1-20.

In some embodiments, the compound of Formula (XI) is a compound of Formula (XIa):

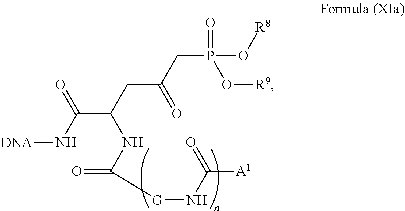

wherein DNA is a nucleic acid barcode;
each instance of G is independently of the formula

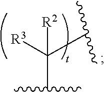

each occurrence of A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
each occurrence of R⁴ is independently selected from the group consisting of H, an optionally substituted C₁-C₁₀ alkyl, and an optionally substituted aryl-(C₁-C₄)alkyl;
each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof:
each occurrence of t is independently an integer of 1-10; and
n is an integer of 1-20.

In various embodiments, the compound for addition to a DEL is a compound of Formula (XVII):

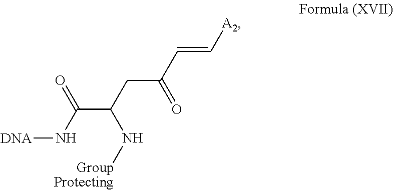

wherein DNA is a nucleic acid barcode;
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In one embodiment, the compound for addition to a DEL is a compound of Formula (XVIII):

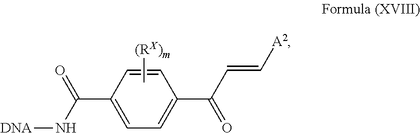

wherein DNA is a nucleic acid barcode;
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each instance of R^{X} is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl; and
wherein m is an integer of 1-4.

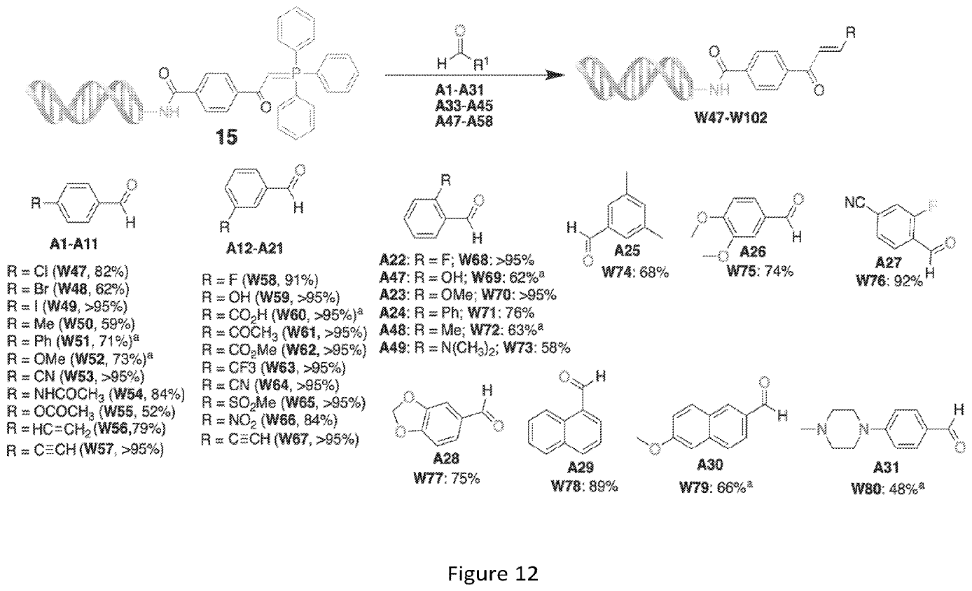

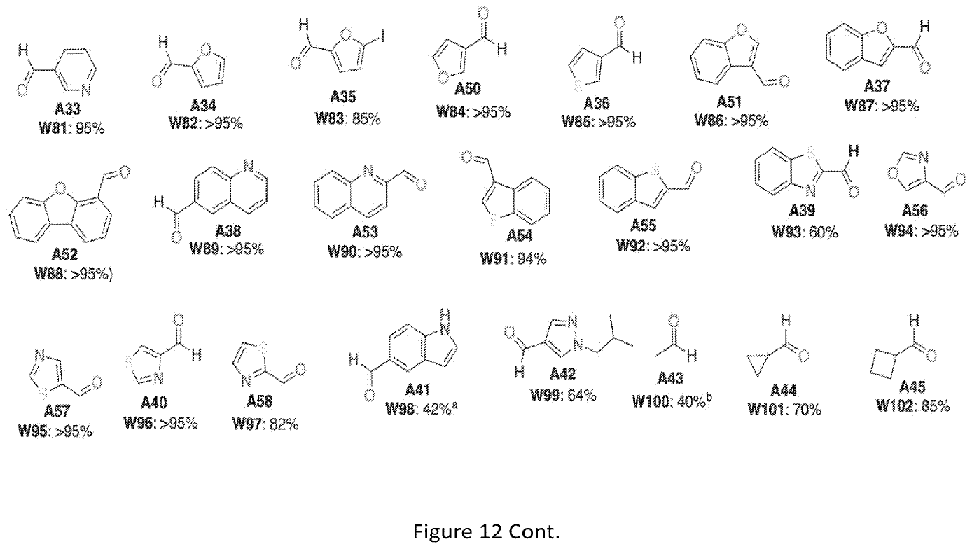

In one embodiment, the compound of Formula (XVIII) has a structure selected from the group consisting of at least one structure shown in FIG. 12.

In various embodiments, the compound for addition to a DEL is a compound of Formula (XIX):

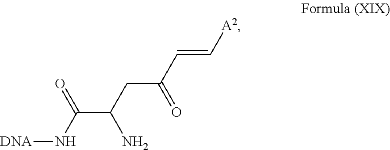

wherein DNA is a nucleic acid barcode; and
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In one embodiment, the compound for addition to a DEL is a compound of Formula (XX):

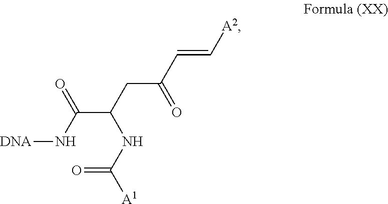

wherein DNA is a nucleic acid barcode; and
each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In one embodiment, the compound for addition to a DEL is a compound of Formula (XXI):

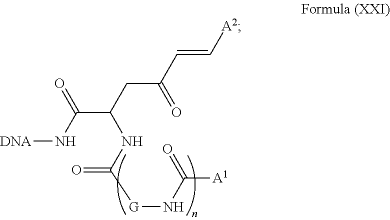

wherein each instance of G is independently of the formula

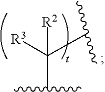

each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R², or R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

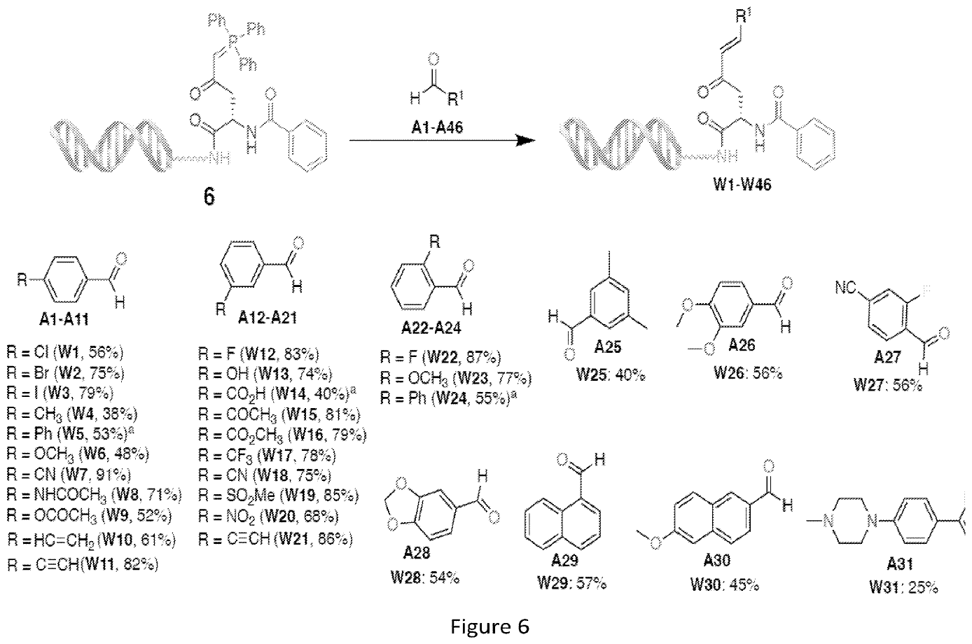

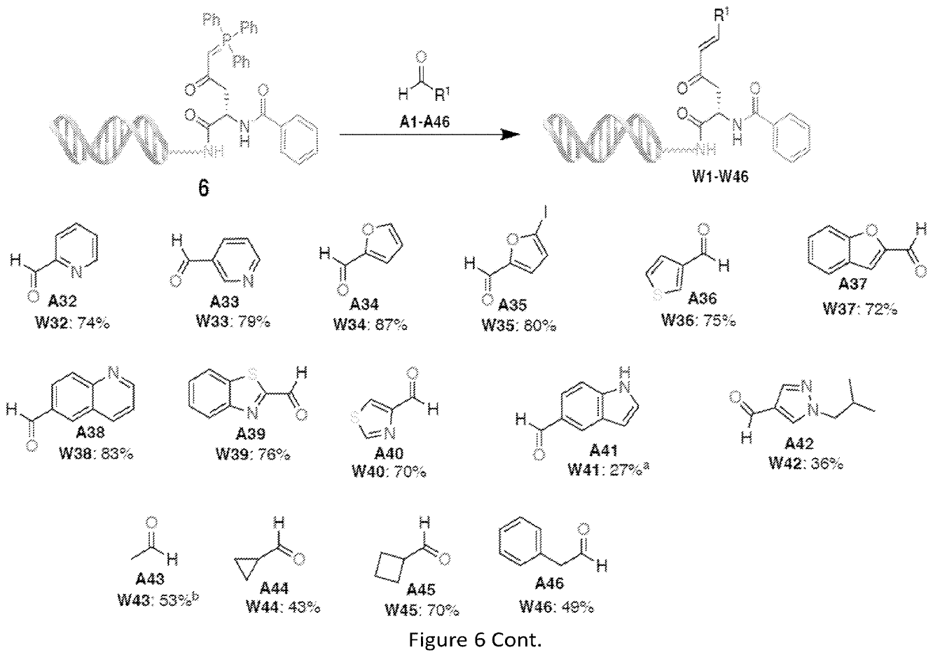

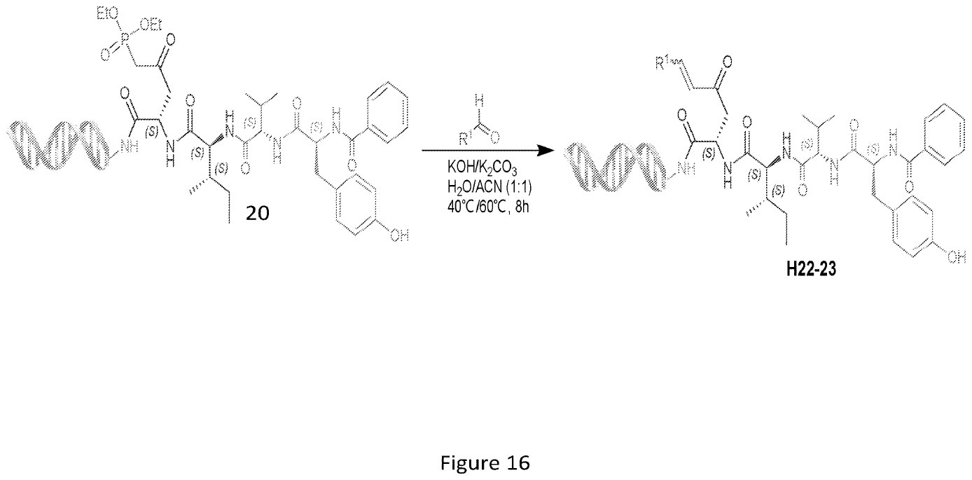

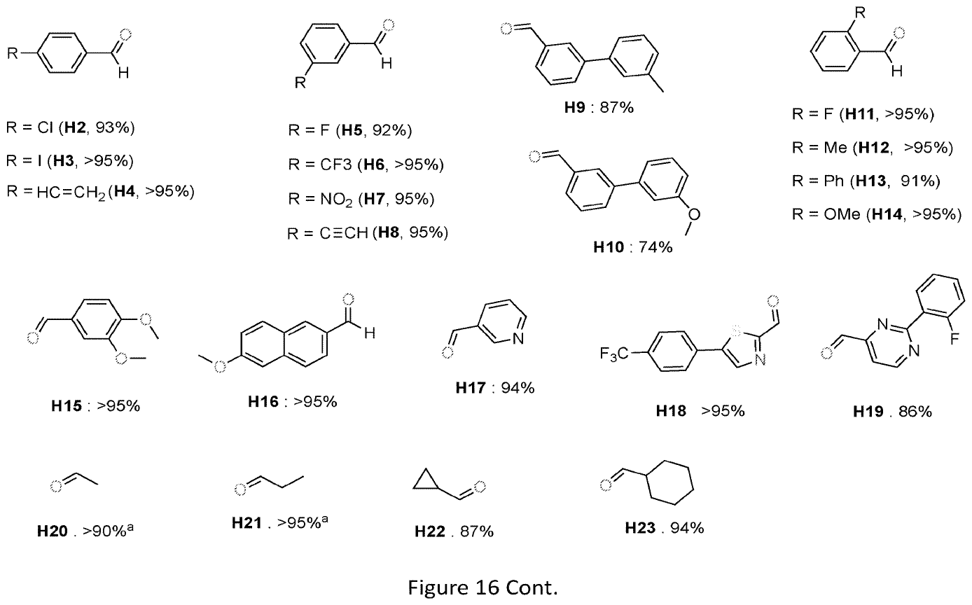

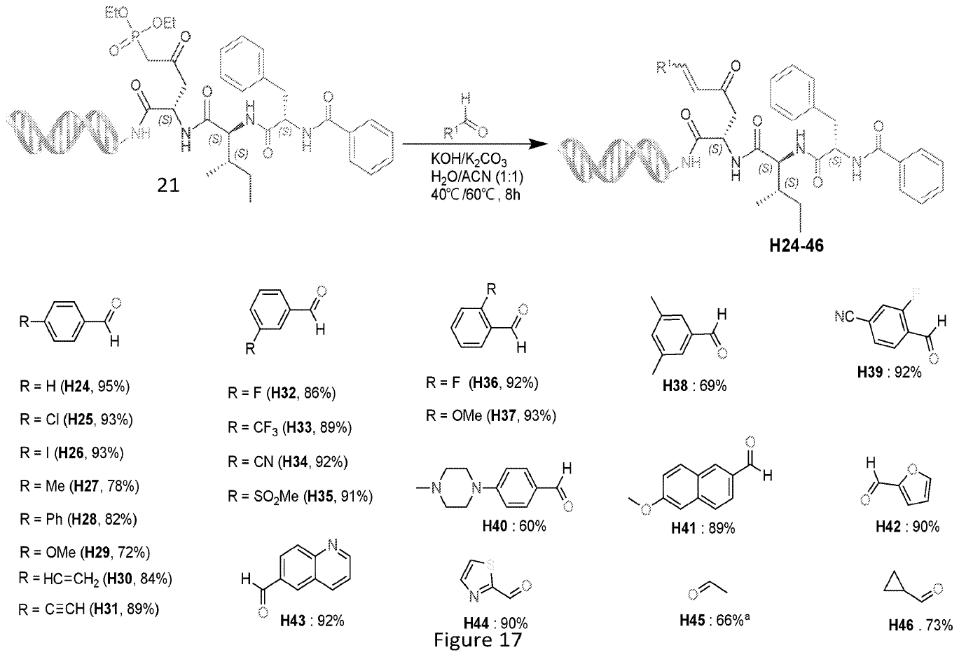

In one embodiment, the compound for addition to a DEL is a has a structure selected from the group consisting of at least one structure as shown in FIG. 6 through FIG. 8, FIG. 16, and FIG. 17.

In some embodiments, the compound for addition to a DEL is a compound of Formula (XXII):

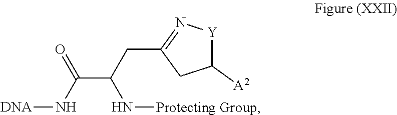

wherein DNA is a nucleic acid barcode;
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
Y is selected from the group consisting of OH, SH, and NHR⁴;
each occurrence of R⁴ is independently selected from the group consisting of H, an optionally substituted C₁-C₁₀ alkyl, and an optionally substituted aryl-(C₁-C₄)alkyl; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, the compound for addition to a DEL is a compound of Formula (XXIII):

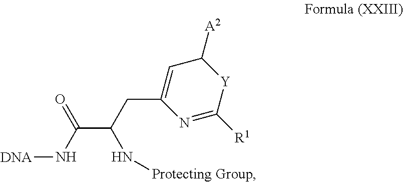

wherein DNA is a nucleic acid barcode;
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R¹ is selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴;
each occurrence of R⁴ is independently selected from the group consisting of H, an optionally substituted C₁-C₁₀ alkyl, and an optionally substituted aryl-(C₁-C₄)alkyl; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

In some embodiments, the compound for addition to a DEL is a compound of Formula (XXIV):

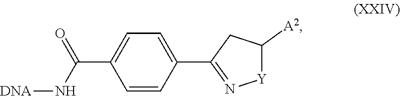

wherein DNA is a nucleic acid barcode;
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
Y is selected from the group consisting of OH, SH, and NHR⁴; and
each occurrence of R⁴ is independently selected from the group consisting of H, an optionally substituted C₁-C₁₀ alkyl, and an optionally substituted aryl-(C₁-C₄)alkyl.

In some embodiments, the compound for addition to a DEL is a compound of Formula (XXV):

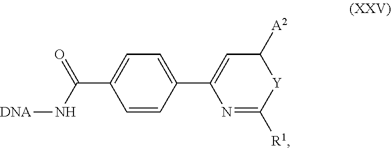

wherein DNA is a nucleic acid barcode;
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R¹ is selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴; and
each occurrence of R⁴ is independently selected from the group consisting of H, an optionally substituted C₁-C₁₀ alkyl, and an optionally substituted aryl-(C₁-C₄)alkyl.

In some embodiments, the compound for addition to a DEL is a compound of Formula (XXVI):

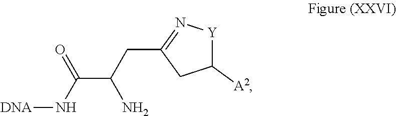

wherein DNA is a nucleic acid barcode:
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
Y is selected from the group consisting of OH, SH, and NHR⁴; and
each occurrence of R⁴ is independently selected from the group consisting of H, an optionally substituted C₁-C₁₀ alkyl, and an optionally substituted aryl-(C₁-C₄)alkyl.

In some embodiments, the compound for addition to a DEL is a compound of Formula (XXVII):

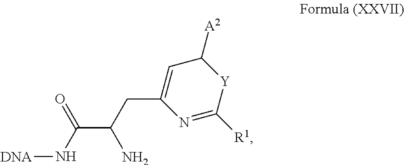

wherein DNA is a nucleic acid barcode;
each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R¹ is selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴; and
each occurrence of R⁴ is independently selected from the group consisting of H, an optionally substituted C₁-C₁₀ alkyl, and an optionally substituted aryl-(C₁-C₄)alkyl.

In some embodiments, the compound for addition to a DEL is a compound of Formula (XXVIII):

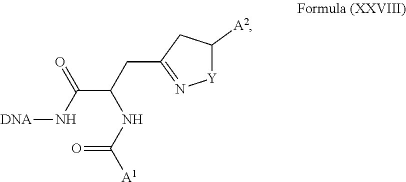

wherein DNA is a nucleic acid barcode;
each occurrence of A¹ and A² is independently selected from the group consisting of H, halo, amino, thio, hydroxyl. C₁-C₁₀ alkyl, C₁-C₁₀ alkenyl. C₁-C₁₀ alkynyl, C₁-C₁₀ alkoxy. C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
Y is selected from the group consisting of OH, SH, and NHR⁴; and
each occurrence of R⁴ is independently selected from the group consisting of H, an optionally substituted C₁-C₁₀ alkyl, and an optionally substituted aryl-(C₁-C₄)alkyl.

In some embodiments, the compound for addition to a DEL is a compound of Formula (XXIX):

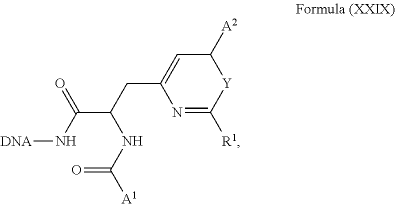

wherein DNA is a nucleic acid barcode;
each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R¹ is selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴; and
each occurrence of R⁴ is independently selected from the group consisting of H, an optionally substituted C₁-C₁₀ alkyl, and an optionally substituted aryl-(C₁-C₄)alkyl.

In some embodiments, the compound for addition to a DEL is a compound of Formula (XIII):

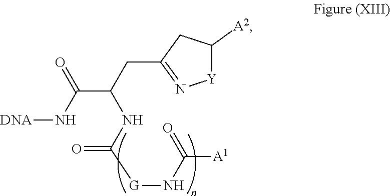

wherein DNA is a nucleic acid barcode;
each instance of G is independently of the formula

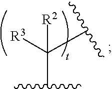

each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

In some embodiments, the compound for addition to a DEL is a compound of Formula (XIV):

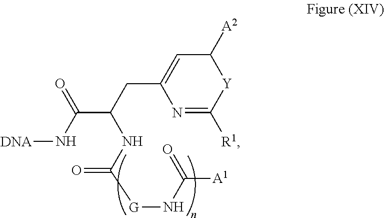

wherein DNA is a nucleic acid barcode;
each instance of G is independently of the formula

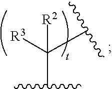

each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R¹, R², and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

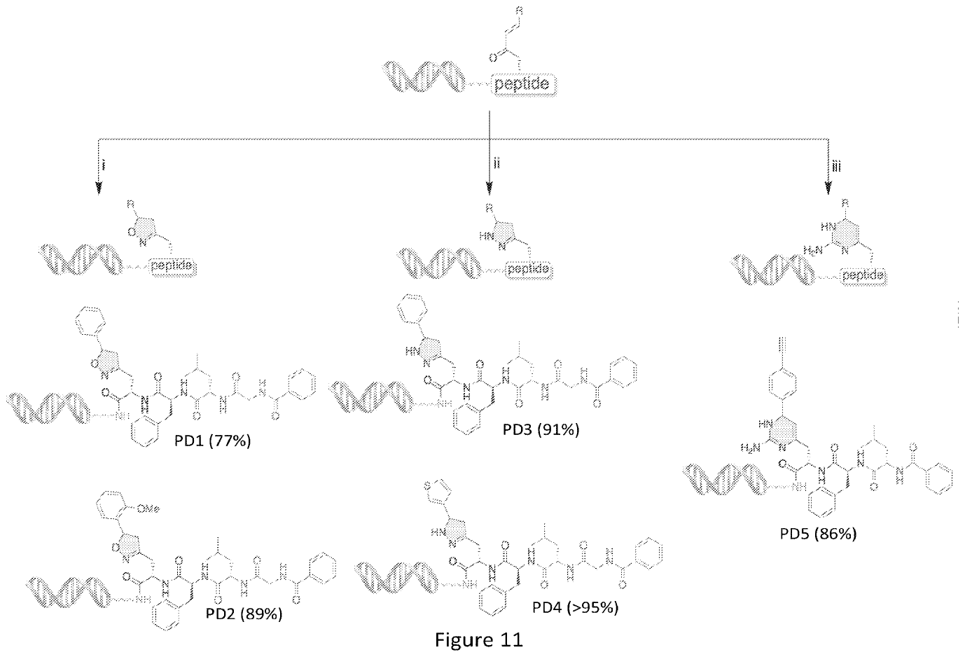

In one embodiment, the compound for addition to a DEL has a structure selected from the group consisting of at least one structure as shown in FIG. 11.

### METHODS

This invention is based, in part, on compounds developed to allow on-DNA reactions that can yield a diverse population of molecules pre-labeled with a DNA barcode for improved preparation efficiency of DNA encoded libraries (DELs).

In one embodiment, the method of preparing compounds for DELs comprises (a) obtaining a DNA barcode; and (b) preparing a phosphonium salt from a phosphorane ylide. In some embodiments, the method comprises a step (c) conjugating the phosphonium salt of step (b) to the DNA barcode of step (a) to yield an on-DNA phosphonium ylide.

In one embodiment, the method of preparing compounds for DELs comprises (a) obtaining a DNA barcode; and (b) preparing a β-keto phosphonate. In some embodiments, the method comprises a step (c) conjugating the β-keto phosphonate of step (b) to the DNA barcode of step (a) to yield an on-DNA β-keto phosphonate.

In some embodiments, the method further comprises step (d) deprotecting an on-DNA phosphonium ylide or on-DNA β-keto phosphonate. In some embodiments, the method further comprises step (e) conjugating a carboxylic acid, including but not limited to an amino acid, to the deprotected on-DNA phosphonium ylide or deprotected on-DNA β-keto phosphonate. In one embodiment, the method further comprises one or more steps (f) repeating steps (d) and (e) with carboxylic acids of interest being added to a deprotected amine of a previously added carboxylic acid to prepare a peptide or peptidomimetic.

In one embodiment, the method further comprises step (g) reacting a phosphorane ylide or β-keto phosphonate with an aldehyde to yield the corresponding α,β-unsaturated ketone. In some embodiments, the method further comprises step (h) reacting α,β-unsaturated ketone with a nucleophilic compound.

In one embodiment, step (b) comprises the following steps: an N-protected Asp-OtBu, a peptide coupling reagent, and a compound of Formula (XXX) are added to an organic solvent, a base is added and stirred, and after purification a compound of Formula (I) is obtained,
wherein the compound of Formula (XXX) is:

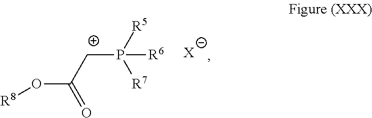

and
wherein the compound of formula (XXXI) is:
wherein each occurrence of R⁵, R⁶, R⁷, R⁸, R⁹, R¹⁰, and R¹¹ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
X is selected from the group consisting of Cl, Br, I, BF₄, acetate, hexafluorophosphate, tosylate, and triflate;
the compound of Formula (I) and an acid are added to a solvent and stirred, and after purification a compound of Formula (II) is obtained.

In one embodiment, step (b) comprises the following steps: an N-protected Asp-OtBu, a peptide coupling reagent, and a compound of Formula (XXX) are added to an organic solvent, a base is added and stirred, and after purification a compound of Formula (I) is obtained; the compound of Formula (I) and an acid are added to a solvent and stirred, and after purification a compound of Formula (II) is obtained.

Examples of N-protected Asp-OtBu include, but are not limited to, Fmoc-Asp-OtBu, Boc-Asp-OtBu, Cbz-Asp-OtBu, Ac-Asp-OtBu, benzylideneamine-Asp-OtBu, Bn-Asp-OtBu, phthalimide-Asp-OtBu, Tr-Asp-OtBu, trifluoroacetamide-Asp-OtBu, and Ts-Asp-OtBu.

Examples of peptide coupling reagents include, but are not limited to, diphenyl phosphoryl azide (DPPA), 1-chloro-N,N,2-trimethyl-1-propenylamine, chloro-N,N,N′,N′-bis(tetramethylene)formamidinium tetrafluoroborate, PyClU, Chloro-N,N,N′,N′-tetramethylformamidinium hexafluorophosphate, Fluoro-N,N,N′,N′-tetramethylformamidinium hexafluorophosphate (TFFH), Fluoro-N,N,N′,N′-bis(tetramethyl)formamidinium hexafluorophosphate (BTFFH), phosgene, triphosgene, thiophosgene, N,N-dicyclohexylcarbodiimide (DCC), N-(3-dimethylaminopropyl)-N′-ethylcarbodiimide (EDC), 1-[3-(dimethylamino)propyl]-3-ethylcarbodiimide methiodide (EDC methiodide), N,N′-diisopropylcarbodiimide (DIC), 1-tert-butyl-3-ethylcarbodiimide (BEC), N-cyclohexyl-N′-(2-morpholinoethyl)carbodiimide metho-p-toluenesulfonate (CMC), N,N′-di-tert-butylcarbodiimide, 1,3-di-p-tolvlcarbodiimide, 1,1′-carbonyldiimidazole (CDI). 1,1′-carbonyl-di-(1,2,4-triazole) (CDT), oxalic acid diimidazolide, 2-chloro-1,3-dimethylimidazolidinium chloride (DMC), 2-chloro-1,3-dimethylimidazolidinium tetrafluoroborate (CIB), 2-chloro-1,3-dimethylimidazolidinium hexafluorophosphate (CIP), 2-fluoro-1,3-dimethylimidazolidinium hexafluorophosphate (DFIH), (benzotriazole-1-yloxy)tris(dimethylamino)phosphonium hexafluorophosphate (BOP), (benzotriazole-1-vloxy)-tripyrrolidinophosphonium hexafluorophosphate (PyBOP)®, (7-azabenzotriazole-1-yloxy)tripyrrolidinophosphonium hexafluorophosphate (PyAOP), 30 bromotris(dimethylamino)phosphonium hexafluorophosphate (BroP), chlorotripyrrolidinophosphonium hexafluorophosphate (PyCloP), bromotripyrrolidinophosphonium hexafluorophosphate (PyBroP®), 3-(diethoxyphosphoryloxy)-1,2,3-benzotriazin-4(3H)-one (DEPBT), O-(benzotriazole-1-yl)-N,N,N′,N′-tetramethyluronium hexafluorophosphate (HBTU), 0-(benzotriazole-1-yl)-N,N,N′,N′-tetramethyluronium tetrafluoroborate (TBTU), 0-(7-azabenzotriazol-1-yl)-N,N,N′,N′-tetramethyluronium hexafluorophosphate (HATU), O-(benzatriazol-1-yl)-N,N,N′,N′-bis(tetramethylene)uronium hexafluorophosphate (HBPyU), O-(benzatriazol-1-yl)-N,N,N′,N′-bis(pentamethylene)uronium hexafluorophosphate (HBPipU), (benzotriazole-1-yloxy)dipiperidinocarbenium tetrafluoroborate (TBPipU), O-(6-chlorobenzotriazol-1-yl)-N,N,N′,N′-tetramethyluronium hexafluorophosphate (HCTU), O-(6-chlorobenzotriazol-I-yl)-N,N,N′,N′-tetramethyluronium tetrafluoroborate (TCTU), O-(3,4-dihydro-4-oxo-1,2,3-benzotriazin-3-yl)-N,N,N′,N′-tetramethyluronium tetrafluoroborate (TDBTU), O-(2-oxo-1(2H)pyridyl)-N,N,N′,N′-tetramethyluronium tetrafluoroborate (TPTU), O-[(etoxycarbonyl)cyanomethylenamino]-N,N,N′,N′-tetramethyluronium hexafluorophosphate (HOTU), O-[(etoxycarbonyl)cyanomethylenamino]-N,N,N′,N′-tetramethyluronium tetrafluoroborate (TOTU), N,N,N′N′-tetramethyl-O—(N-succinimidyl)uronium hexafluorophosphate (HSTU), N,N,N′N′-tetramethyl-O—(N-succinimidyl)uronium tetrafluoroborate (TSTU), dipyrrolidino(N-succinimidyloxy)carbenium hexafluorophosphate (HSPyU), S-(1-oxido-2-pyridyl)-N,N,N′,N′-tetramethyluronium tetrafluoroborate (TOTT), propylphosphonic anhydride, 2-chloro-1-methylpyridinium iodide. 2-chloro-4,6-dimethyoxy-1,3,5-triazine (CDMT), 4-(4,6-dimethoxy-1,3,5-triazin-2-yl)-4-methylmorpholinium chloride (DMTMM), hydroxybenzotriazole (HOBt), and 1-hydroxy-7-azabenzotriazole (HOAt).

In various embodiments, step (b) comprises the following steps: Fmoc-Asp-OtBu, BTFFH, and a compound of Formula (XXIII) are added to dry CH₂Cl₂, DIPEA is added, and stirred at room temperature for 8-24 hours, CH₂Cl₂ is removed, and after purification a compound of Formula (I) is obtained, the compound of Formula (I) and trifluoroacetic acid (TFA) are added to dry CH₂Cl₂, stirred at room temperature for 4 hours. CH₂Cl₂ is removed, and after purification a compound of Formula (II) is obtained.

In one embodiment, the compound of Formula (XXX) is

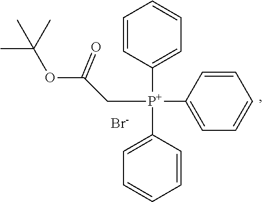 [SMILES: CC(C)(C)OC(=O)C[P+](c1ccccc1)(c1ccccc1)c1ccccc1 ; [Br-]]

yielding

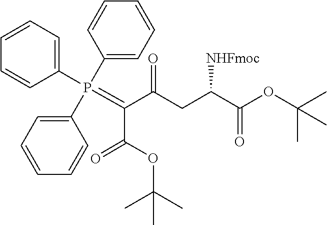

as the compound of Formula (I) and the compound of Formula (II) is

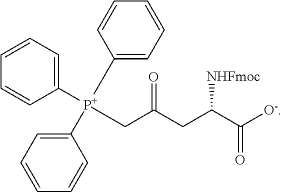

In one embodiment, step (b) comprises the following steps: 4-(tert-butoxycarbonyl)benzoic acid, BTFFH, and (2-(tert-butoxy)-2-oxoethyl)triphenylphosphonium bromide are added to dry CH₂Cl₂, DIPEA is added, and stirred at room temperature for 8-24 hours, CH₂Cl₂ is removed, and after purification

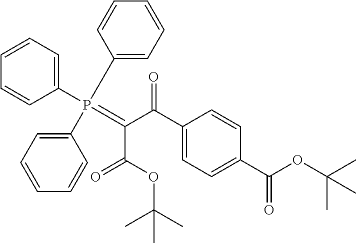 [SMILES: CC(C)(C)OC(=O)C(C(=O)c1ccc(C(=O)OC(C)(C)C)cc1)=P(c1ccccc1)(c1ccccc1)c1ccccc1]

is obtained,

TFA and

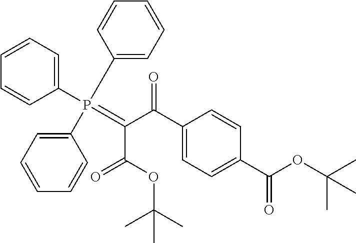 [SMILES: CC(C)(C)OC(=O)C(C(=O)c1ccc(C(=O)OC(C)(C)C)cc1)=P(c1ccccc1)(c1ccccc1)c1ccccc1]

are added to dry CH₂Cl₂, stirred at room temperature for 4 hours. CH₂Cl₂ is removed, and after purification

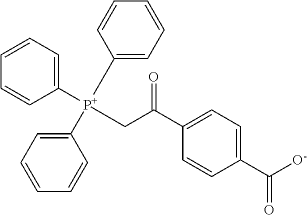 [SMILES: O=C([O-])c1ccc(C(=O)C[P+](c2ccccc2)(c2ccccc2)c2ccccc2)cc1]

is obtained.

In some embodiments, step (b) comprises the following steps: (i) a compound of Formula (XXXI) and a compound of Formula (XXXII) are added to an organic solvent, a base is added and stirred, the base quenched, and solvent removed to yield the product; (ii) the product of (i) is stirred in an organic solvent with trifluoroacetic acid (TFA) and the solvent removed to yield the product; and (iii) the product of (ii) is added to a protecting agent in a basic aqueous solution, stirred, the solvent removed, and purified, yielding a compound of Formula (IV),
wherein the compound of Formula (XXXI) is:

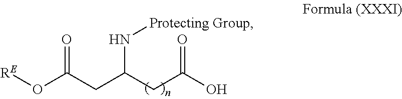

wherein R^{E} is selected from the group consisting of C₁-C₁₀ alkyl and C₃-C₁₀ cycloalkyl;
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl; and
n=1-10; and
wherein the compound of Formula (XXXII) is:

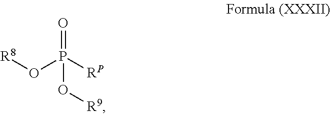

wherein R⁸, R⁹, and R^{P} are each independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof. Examples of bases useful in step (i) include, but are not limited to, lithium diisopropylamide (LDA), sodium diisopropylamide (NaDA), lithium bis(trimethylsilyl)amide (LiHMDS), sodium bis(trimethylsilyl)amide (NaHMDS), lithium diethylamide, sodium diethylamide, lithium 2,2,6,6-tetramethylpiperidide (LiTMP), and sodium 2,2,6,6-tetramethylpiperidide (NaTMP).

In some embodiments, the composition of Formula (XXXI) is an N-protected Asp-OR^{E}. Examples of N-protected Asp-OR^{E} include, but are not limited to, Fmoc-Asp-OMe, Boc-Asp-OMe, Cbz-Asp-OMe, Ac-Asp-OMe, benzylideneamine-Asp-OMe, Bn-Asp-OMe, phthalimide-Asp-OMe. Tr-Asp-OMe, trifluoroacetamide-Asp-OMe, Ts-Asp-Ome Fmoc-Asp-OEt, Boc-Asp-OEt, Cbz-Asp-OEt. Ac-Asp-OEt, benzylideneamine-Asp-OEt, Bn-Asp-OEt, phthalimide-Asp-OEt, Tr-Asp-OEt, trifluoroacetamide-Asp-OEt, Ts-Asp-OEt, Fmoc-Asp-O(nPr), Boc-Asp-O(nPr), Cbz-Asp-O(nPr), Ac-Asp-O(nPr), benzylideneamine-Asp-O(nPr), Bn-Asp-O(nPr), phthalimide-Asp-O(nPr), Tr-Asp-O(nPr), trifluoroacetamide-Asp-O(nPr). Ts-Asp-O(nPr) Fmoc-Asp-O(iPr), Boc-Asp-O(iPr), Cbz-Asp-O(iPr), Ac-Asp-O(iPr), benzylideneamine-Asp-O(iPr), Bn-Asp-O(iPr), phthalimide-Asp-O(iPr), Tr-Asp-O(iPr), trifluoroacetamide-Asp-O(iPr), Ts-Asp-O(iPr), Fmoc-Asp-O(tBu), Boc-Asp-O(tBu), Cbz-Asp-O(tBu), Ac-Asp-O(tBu), benzylideneamine-Asp-O(tBu), Bn-Asp-O(tBu), phthalimide-Asp-O(tBu), Tr-Asp-O(tBu), trifluoroacetamide-Asp-O(tBu), and Ts-Asp-O(tBu).

In some embodiments, the compound of Formula (XXXII) is a methylphosphonate. Examples of methylphosphonate include, but are not limited to, dimethyl methylphosphonate, diethyl methylphosphonate, ethyl, methyl methylphosphonate, dipropyl methylphosphonate, and diisopropyl methylphosphonate.

In various embodiments, step (b) comprises the following steps: (i) a compound of Formula (XXXI) and a compound of Formula (XXXII) are added to dry THF, LDA is added 0° C., the reaction stirred for 15 minutes, the reaction quenched, solvent removed, and the product isolated; (ii) the product if step (i) is stirred in 20% TFA in CH₂Cl₂ for one hour, and the solvent is removed; and (iii) the product of step (ii) is stirred in water/acetone with Fmoc succinate (Fmoc-OSu) and NaHCO₃, the solvent is removed, and after purification a compound of Formula (VI) is obtained.

In one embodiment, the compound of Formula (XXXI) is Boc-Asp-OMe, and the compound of Formula (XXXII) is diethyl methylphosphonate, and the compound of Formula (II) is

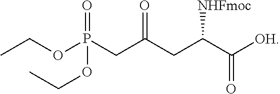

In one embodiment, step (c) comprises the following steps: a DNA barcode is suspended in an aqueous solution, a compound of Formula (II) or a compound of Formula (IV), one or more peptide coupling reagent, and a base are added and stirred, the DNA-conjugate precipitated, and the solvent removed, yielding a compound of Formula (III) or Formula (V), respectively.

In various embodiments, step (c) comprises the following steps: a DNA barcode is suspended in a pH 8.0 aqueous solution of 3-(N-morpholino)propanesulfonic acid (MOPS) and sodium chloride (NaCl), a compound of Formula (II) or Formula (IV), EDC, and HOAt are added, DIPEA in dimethyl sulfoxide (DMSO) is added and stirred at 20-25° C. for 2-4 hours, aqueous NaCl and ethanol (EtOH) are added and stored at −80° C. for 1 hour, the solution is centrifuged, the supernatant is removed, the pellet is washed and dried, yielding a compound of Formula (III) is obtained.

In one embodiment, the compound of Formula (II) is

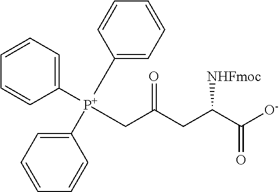

and the compound of Formula (III) is a compound of Formula (IIIa).

In one embodiment, the compound of Formula (IV) is

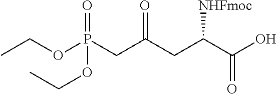

and the compound of formula (V) is a compound of Formula (Va).

In one embodiment, step (c) comprises the following steps: a DNA barcode is suspended in a pH 8.0 aqueous solution of MOPS and NaCl,

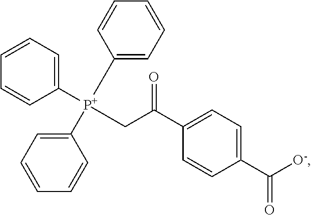

EDC, and HOAt are added, DIPEA in DMSO is added and stirred at 25° C. for 4 hours, aqueous NaCl and EtOH are added and stored at −80° C. for 1 hour, the solution is centrifuged, the supernatant is removed, the pellet is washed and dried, yielding a compound of Formula (IV).

In some embodiments, step (c) conjugates the compound of Formula (II) or Formula (IV) to a primary amine of the DNA barcode. In some embodiments, step (c) conjugates the compound of Formula (II) or Formula (IV) to a primary amine of an unpaired nucleotide of a single strand overhang in the DNA barcode. In some embodiments, step (c) conjugates the compound of Formula (II) or Formula (IV) to a primary amino of a linker conjugated to the DNA barcode. In some embodiments, the linker is a polyethylene glycol (PEG) linker.

In one embodiment, step (d) comprises the following steps: a compound of Formula (III) or Formula (V) is added to an aqueous solution, a DNA barcode-conjugate-compatible reagent for removing a protecting group added and stirred, the DNA barcode-conjugate precipitated, and the solvent removed, yielding a compound of Formula (VI) or Formula (VII), respectively.

In various embodiments, step (d) comprises the following steps: a compound of Formula (III) or Formula (V) is added to water, a solution of 20% piperidine in water is added and stirred at 25° C. for 24 hours, aqueous NaCl and EtOH are added and stored at −80° C. for 1 hour, the solution is centrifuged, the supernatant is removed, the pellet is washed and dried, yielding a compound of Formula (VI) or Formula (VII), respectively.

In one embodiment, the compound of Formula (III) is a compound of Formula (IIIa), yielding a compound of Formula (VIa). In one embodiment, the compound of Formula (V) is a compound of Formula (Va), yielding a compound of Formula (VIIa).

In one embodiment, step (e) comprises the following steps: a compound of Formula (VI) or Formula (VII) is suspended in an aqueous solution, a compound of Formula (XXXIII), one or more peptide coupling reagents, and a base added and stirred, the DNA-conjugate precipitated, and the solvent removed, yielding a compound of Formula (VIII) or Formula (IX), respectively,
wherein the compound of Formula (XXXIII)

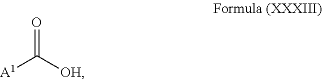

is
wherein each occurrence of A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In a variety of embodiments, the compound of Formula (XXXIII) is an amino acid, a non-naturally encoded amino acid, a protected amino acid, a protected non-naturally encoded amino acid

In various embodiments, step (e) comprises the following steps: a compound of Formula (VI) or Formula (VII) is suspended in a pH 8.0 aqueous solution of MOPS and NaCl, a compound of Formula (XXXIII), EDC, and HOAt are added, DIPEA in DMSO is added and stirred at 25° C. for 4 hours, aqueous NaCl and ethanol (EtOH) are added and stored at −80° C. for 1 hour, the solution is centrifuged, the supernatant is removed, the pellet is washed and dried, yielding a compound of Formula (VIII) or Formula (IX), respectively, is obtained.

In some embodiments, the compound of Formula (VI) is a compound of Formula (VIa), yielding a compound of Formula (VIIIa). In some embodiments, the compound of Formula (VII) is a compound of Formula (VIIa), yielding a compound of formula (IXa).

In various embodiments, step (f) comprises one or more additional steps (d) and (e) adding units of Formula (XXXIII). Step (f) can comprise 1, 2, 3, 4, 5, 6, 7, 8, 9, 10, 11, 12, 13, 14, 15, 16, 17, 18, 19, or 20 additional cycles of (d) and (e). In some embodiments, an additional step (d) is appended. Examples of step (f) include, but are not limited to: (d), (e); (d), (e), (d); ((d), (e))×2; ((d), (e))×2, (d); ((d), (e))×3; ((d), (e))×3, (d); ((d), (e))×4; ((d), (e))×4, (d); ((d), (e))×5; ((d), (e))×5, (d); ((d), (e))×10; ((d), (e))×10, (d); ((d), (e))×15; ((d), (e))×15, (d); ((d), (e))×20; ((d), (e))×20, (d).

In various embodiments, the method includes a step (g). Step (g) can take place after a step (c), (d), (e), or (f).

In one embodiment, step (g) comprises the following steps: a compound of Formula (III) or (V), Formula (XV) or (XVI), Formula (VI) or (VII), Formula (VIII) or (IX), Formula (X) or (XI), or Formula (XV) or (XVI) is added to an aqueous solution, a compound of Formula (XXXIV) is added, a base in a co-solvent is added and stirred, the DNA-conjugate precipitated, and the solvent removed, yielding a compound of Formula (XVII), (XVIII), (XIX), (XX), or (XXI) respectively,
wherein the compound of Formula (XXXIV) is:

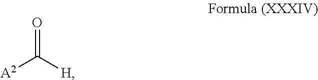

wherein each occurrence of A² is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

In various embodiments, step (g) comprises the following steps: a compound of Formula (III) or Formula (V) is added to water, a compound of Formula (XXXIV) is added, a solution of DIPEA in acetonitrile (CH₃CN) is added and stirred at 50° C. for 12 hours, aqueous NaCl and EtOH are added and stored at −80° C. for 1 hour, the solution is centrifuged, the supernatant is removed, and the pellet is washed and dried, yielding a compound of Formula (XVII).

In some embodiments, step (g) comprises the following steps: a compound of Formula (XV) or Formula (XVI) is added to water, a compound of Formula (XXXIV) is added, a solution of DIPEA in acetonitrile (CH₃CN) is added and stirred at 50° C. for 12 hours, aqueous NaCl and EtOH are added and stored at −80° C. for 1 hour, the solution is centrifuged, the supernatant is removed, and the pellet is washed and dried, yielding a compound of Formula (XVIII).

In some embodiments, step (g) comprises the following steps: a compound of Formula (VI) or Formula (VII) is added to water, a compound of Formula (XXXIV) is added, a solution of DIPEA in acetonitrile (CH₃CN) is added and stirred at 50° C. for 12 hours, aqueous NaCl and EtOH are added and stored at −80° C. for 1 hour, the solution is centrifuged, the supernatant is removed, and the pellet is washed and dried, yielding a compound of Formula (XIX).

In some embodiments, step (g) comprises the following steps: a compound of Formula (VIII) or Formula (IX) is added to water, a compound of Formula (XXXIV) is added, a solution of DIPEA in acetonitrile (CH₃CN) is added and stirred at 50° C. for 12 hours, aqueous NaCl and EtOH are added and stored at −80° C. for 1 hour, the solution is centrifuged, the supernatant is removed, and the pellet is washed and dried, yielding a compound of Formula (XX).

In some embodiments, step (g) comprises the following steps: a compound of Formula (X) or Formula (XI) is added to water, a compound of Formula (XXV) is added, a solution of DIPEA in acetonitrile (CH₃CN) is added and stirred at 50° C. for 12 hours, aqueous NaCl and EtOH are added and stored at −80° C. for 1 hour, the solution is centrifuged, the supernatant is removed, and the pellet is washed and dried, yielding a compound of Formula (XXI).

In some embodiments, the method includes a step (h).

In one embodiment, step (h) comprises the following steps: a compound of Formula (XVII), (XVIII), (XIX), (XX), or (XXI) is suspended in an aqueous solution, a solution of a compound of Formula (XXVI) is added and stirred, the DNA-conjugate is precipitated, and the solvent is removed, yielding a compound of Formula (XXII), (XXIV), (XXVI), (XXVIII), or (XIII), respectively,
wherein the compound of Formula (XXVI) is

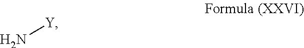

wherein Y is selected from the group consisting of OH, SH, and NHR⁴; and
each occurrence of R⁴ is independently selected from the group consisting of H, an optionally substituted C₁-C₁₀ alkyl, and an optionally substituted aryl-(C₁-C₄)alkyl.

In some embodiments, step (h) comprises the following steps: a compound of Formula (XVII), (XVIII). (XIX), (XX), or (XXI) is suspended in water and added to an aqueous phosphate buffer solution at pH 5.5, an aqueous solution of hydroxylamine hydrochloride is added and stirred at 60° C. for 2 hours, aqueous NaCl and EtOH are added and stored at −80° C. for 1 hour, the solution is centrifuged, the supernatant is removed, and the pellet is rinsed and dried, yielding a compound of Formula (XXII), (XXIV), (XXVI), (XXVIII), or (XIII), respectively,
wherein Y is O in the compound of Formula (XXII), (XXIV), (XXVI), (XXVIII), or (XIII).

In some embodiments, step (h) comprises the following steps: a compound of Formula (XVII), (XVIII), (XIX), (XX), or (XXI) is suspended in water and added to an aqueous borate buffer solution at pH 9.5, an aqueous solution of hydrazine hydrate is added and stirred at 60° C. for 2 hours, aqueous NaCl and EtOH are added and stored at −80° C. for 1 hour, the solution is centrifuged, the supernatant is removed, and the pellet is rinsed and dried, yielding a compound of Formula (XXII), (XXIV). (XXVI), (XXVIII), or (XIII), respectively,
wherein Y is NR and wherein R⁴ is H in the compound of Formula (XXII), (XXIV), (XXVI), (XXVIII), or (XIII).

In one embodiment, step (h) comprises the following steps: a compound of Formula (XVII), (XVIII), (XIX), (XX), or (XXI) is suspended in an aqueous solution, a solution of a compound of Formula (XXXVI) is added and stirred, the DNA-conjugate is precipitated, and the solvent is removed, yielding a compound of Formula (XXIII), (XXV), (XXVII), (XXIX), or (XIV), respectively,
wherein the compound of Formula (XXXVI) is

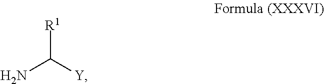

wherein each occurrence of R¹ is selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NHR⁴; and
each occurrence of R⁴ is independently selected from the group consisting of H, an optionally substituted C₁-C₁₀ alkyl, and an optionally substituted aryl-(C₁-C₄)alkyl.

In some embodiments, step (h) comprises the following steps: a compound of Formula (XVII), (XVIII), (XIX), (XX), or (XXI) is suspended in water and added to an aqueous buffer solution, an aqueous solution of a compound of Formula (XXXVI) is added and stirred at 60-80° C. for 2-6 hours, aqueous NaCl and EtOH are added and stored at −80° C. for 1 hour, the solution is centrifuged, the supernatant is removed, and the pellet is rinsed and dried, yielding a compound of Formula (XXIII), (XXV), (XXVII), (XXIX), or (XIV), respectively.

In some embodiments, step (h) comprises the following steps: a compound of Formula (XVII), (XVIII), (XIX), (XX), or (XXI) is suspended in water and added to an aqueous borate buffer solution at pH 9.5, an aqueous solution of guanidine hydrochloride is added and stirred at 70° C. for 3 hours, aqueous NaCl and EtOH are added and stored at −80° C. for 1 hour, the solution is centrifuged, the supernatant is removed, and the pellet is rinsed and dried, yielding a compound of Formula (XXIII), (XXV), (XXVII), (XXIX), or (XIV), respectively,
wherein Y is NR⁴, wherein R⁴ is H, and wherein one instance of R¹ is NH₂ in the compound of Formula (XXIII), (XXV). (XXVII), (XXIX), or (XIV).

The methods of the present invention comprise multiple steps (a)-(h), and it is envisioned that not all embodiments of the method will comprise all steps listed. In some embodiments of the method steps may be skipped. Examples of methods according to the present invention include, but are not limited to: (a), (b); (a), (b), (c); (a), (b), (c), (d); (a), (b), (c), (d), (e); (a), (b), (c), (d), (e), (g); (a), (b), (c), (d), (e), (g), (h); (a), (b), (c), (d), (e), (f), (g); (a), (b), (c), (d), (e), (f), (g), (h); (a), (b), (c), (d), (e), (f)×2, (g), (h); (a), (b), (c), (g); (a), (b), (c), (g), (h); (a), (b), (c), (d), (g); (a), (b), (c), (d), (e), (f)×5, (g), (h).

In other embodiments, compounds of interest may be acquired from alternate sources for use in the method. For example, it is conceivable that the phosphorane ylide that would result from step (a), the phosphonium salt that would result from step (b), or a pre-made on-DNA phosphorane ylide of step (c) or (d) may be purchased commercially. As such, in some embodiments, the methods of may begin from any point in the list of steps. Examples of methods according to the present invention include, but are not limited to: (b); (b), (c); (b), (c), (d); (b), (c), (d), (e); (b), (c), (d), (e), (g); (b), (c), (d), (e), (g), (h); (b), (c), (d), (e), (f), (g); (b), (c), (d), (e), (f), (g), (h); (b), (c), (d), (e), (f)×2, (g), (h); (b), (c), (g); (b), (c), (g), (h); (b), (c), (d), (g); (c); (c), (d); (c), (d), (e); (c), (d), (e), (g); (c), (d), (e), (g), (h); (c), (d), (e), (f), (g); (c), (d), (e), (f), (g). (h); (c), (d), (e), (f)×2, (g), (h); (c), (g); (c), (g), (h); (c), (d). (g); (d); (d), (e); (d), (e), (g); (d), (e), (g), (h); (d), (e), (f), (g); (d), (e), (f), (g), (h); (d), (e), (f)×2, (g), (h); (g); (g), (h); (e); (e), (f); (e), (f), (g); (e), (f), (g), (h); (e), (g); and (e), (g), (h).

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: DNA-Compatible Wittig Olefination of on-DNA Peptidyl Ylides

Amino acids are widely used as building blocks in DNA-encoded libraries due to the compatibility of their functional groups with on-DNA reaction conditions (Franzini, R. M., et al., 2016, Journal of Medicinal Chemistry, 59(14):6629-6644). Amino acids harboring functional groups on their side chain (acidic and basic amino acids) are particularly valuable scaffolds as they allow the introduction of non-native structural diversified blocks in a combinatorial manner. Switching a functional group of a natural amino acid, especially from the sidechain, with a different functional group leads to a new scaffold and contributes to DEL chemical space expansion. Introducing a reactive phosphorus moiety in the sidechains of native amino acids has been reported in standard organic chemistry. Particularly, C-terminal variations on peptides are consistently explored, based on C-acylated phosphoranes using standard solid phase peptide chemistry (Masri, E., et al., 2020, Organic Letters, 22(8):2976-2980). Here, to generate structurally diversified DNA-encoded peptidomimetics, an adapted approach was utilized to generate, in a DNA-compatible manner, on-DNA α,β-unsaturated ketones first and then use these intermediates to perform Diversity-Oriented Synthesis (DOS).

### Generation of Phosphorane-Ylides

Both Aspartic and Glutamic acids can be modified to generate phosphonium-ylides-based amino acids targeting the acid functionality of their side chains. Aspartic acid-based ylides were investigated first to test their compatibility and tolerance with Wittig olefination, and then for peptide elongation. For that, trisubstituted phosphonium salts 1a and 1b (FIG. 1) were synthesized through P-alkylation of triphenyphosphine and diphenylmethylphosphine, respectively, with tert-butyl-2-bromoacetate. N-Fmoc-L-aspartic acid-α-tert-butyl ester was activated using Bis(tetramethylene)-fluoroformamidinium hexafluorophosphate (BTFFH) as a coupling reagent and C-acylated with phosphoranylidene acetates which were generated in situ from the salts 1a and 1b with excess of base DIPEA to provide the phosphorane-ylides 2a and 2b. Both the phosphorane-ylides 2a and 2b were treated with trifluoroacetic acid to provide Fmoc-Asp-derived phosphonium salts 3a and 3b by cleavage of tert-butyl ester and instantaneous decarboxylation (FIG. 1).

### Conjugation of Ylides to DNA

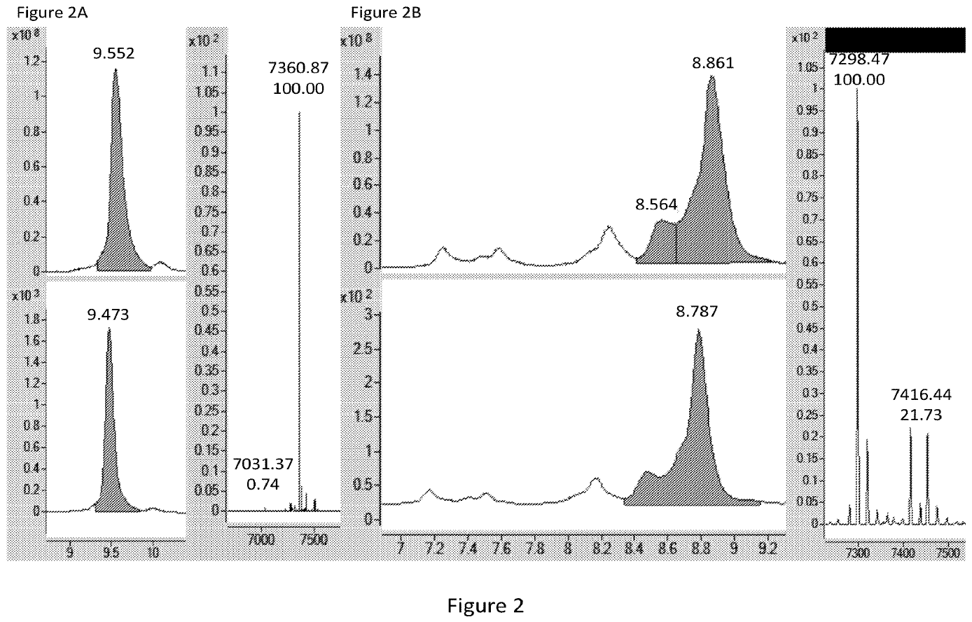

Fmoc-Asp-derived phosphonium salts 3a and 3b were then coupled with DNAI through amidation using EDC/HOAt as coupling reagents in presence of DIPEA in MOPS buffer (pH 8.0, 0.5M NaCl)/DMSO (Li, Y. Z., et al., 2016, ACS Combinatorial Science, 18(8):438-443). When 100 equivalents of EDC/HOAt/DIPEA and phosphonium salt 3a in DMSO were added to DNA1 in MOPS buffer, the mixture precipitated. 3a was then used at 20 equivalents and other reagents at 100 equivalents. After the reaction mixture was stirred (RT, 4 hours), the desired DNA-conjugated-Fmoc-Asp-derived triphenyl phosphorane-ylide 4a was obtained in a quantitative yield (FIGS. 1 and 2). The DNA fragments used throughout were double-strand DNA covalently linked, 8 nucleotide-long, with a single strand overhang of 3 nucleotides and a 5′ phosphate group as previously described reactions (Nielsen, J., et al., 1993, Journal of the American Chemical Society, 115(21):9812-9813; Gartner. Z. J., et al., 2002, Angewandte Chemie International Edition, 41(10):1796-1800; Clark, M. A., et al., 2009; Nature Chemical Biology, 5:647-654; Ding, Y., et al., 2015, ACS Combinatorial Science, 17(1):1-4; Litovchick, A., 2015, Scientific Reports, 5:10916; Li, Y. Z., et al., 2016, ACS Combinatorial Science, 18(8):438-443; Du, H. C., et al., 2017, Bioconjugate Chemistry, 28(10):2575-2580; Lu, X. J., et al., 2017, MedChemComm, 8(8):1614-1617; Skopic, M. K., et al., 2017, Chemical Science, 8:3356-3361; Kolmel, D. K., et al., 2018, ChemMedChem, 13(20):2159-2165; Li, J. Y., et al., 2018, Bioconjugate Chemistry, 29(11):3841-3846; Shu, K., et al., 2018, ACS Combinatorial Science, 20(5):277-281; Wang, J., et al., Proceedings of the National Academy of Sciences USA, 115(28):E6404-E6410; Wang, X., et al., 2018, Organic Letters, 20(16):4764-4768; Zhu, Z., et al., 2018, ACS Chemical Biology, 13(1):53-59; Flood, D. T., et al., 2019, Journal of the American Chemical Society, 141(25):9998-10006; Gerry, C. J., et al., Journal of the American Chemical Society, 141(26):10225-10235; Gironda-Martinez, A., et al., 2019, Organic Letters. 21(23):9555-9558; Kolmel, D. K.. 2019, ACS Combinatorial Science, 21(8):588-597; Kunig, V. B. K., et al., 2019, Organic Letters, 21(18):7238-7243; Li, J. Y., et al., 2019, Bioconjugate Chemistry, 30(8):2209-2215; Phelan, J. P., et al., Journal of the American Chemical Society, 141(8):3723-3732; Wang, X., et al, 2019, Organic Letters, 21(3):719-723; Xu, H. T., et al., 2019, Advanced Science, 6:1901551; Chen, Y. C., et al., 2020, Bioconjugate Chemistry, 31(3):770-780; Fan, Z., et al., 2020. Chemical Science. 31(25):12282-12288; Fitzgerald. P. R., et al., 2020, Chemical Reviews, 121(12):7155-7177; Flood, D. T., et al., 2020, Angewandte Chemie International Edition, 59(19):7377-7383; Kolmel, D. K., et al., 2020, Biochemical and Biophysical Research Communications, 533(2):201-208: Shan, J., et al., 2021, Bioorganic & Medicinal Chemistry, 42:116234; Yang, P., et al., 2021, Chemical Science, 12(16):5804-5810; Adamik, R., 2022, Chemistry, 28(20):e202103967; Eom, S., et al., 2022, Organic Letters, 24(27):4881-4885; Kolusu, S. R. N., et al., 2022, Chemical Science, 13(23):6982-6989; Krumb, M., et al., 2022, Chemical Science, 13(4):1023-1029; Siripuram V. K., 2022, Frontiers in Chemistry. 10:894603; Stanway-Gordon, H. A., et al., 2022, Angewandte Chemie International Edition, 61(3):e202111927; Xu, H. T., et al., Advanced Science, 9(26):2202790; Yen-Pon, E., et al., 2022, Journal of the American Chemical Society, 144(27):12184-12191; Zhong. S., et al., 2022, Organic Letters, 24(4):1022-1026; Satz, A. L., et al., 2015, Bioconjugate Chemistry, 26(8):1623-1632). Similarly, DNA-conjugated phosphorane-ylide 4b was achieved with a quantitative yield (FIGS. 1 and 2).

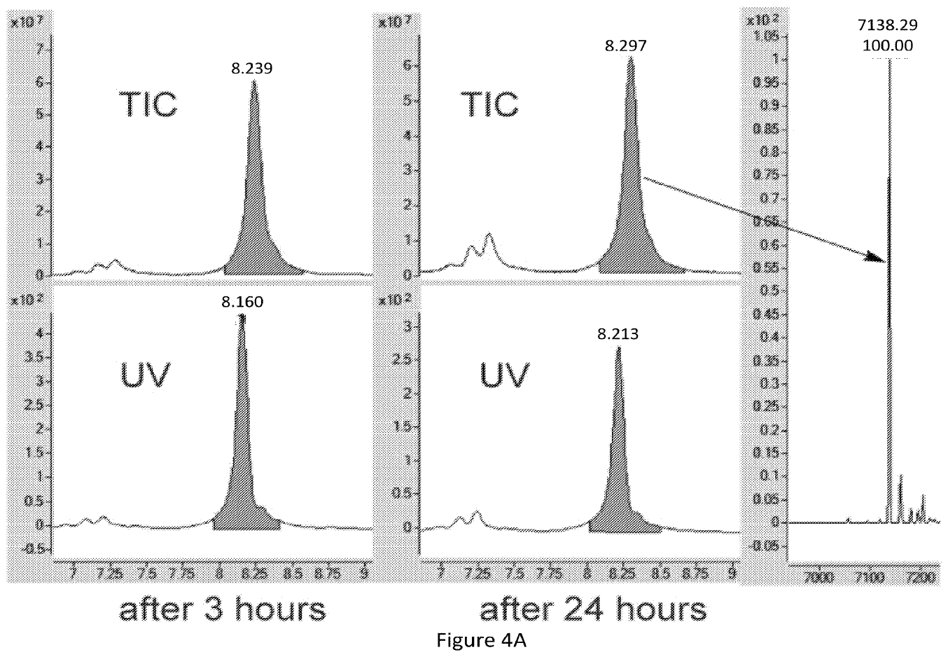

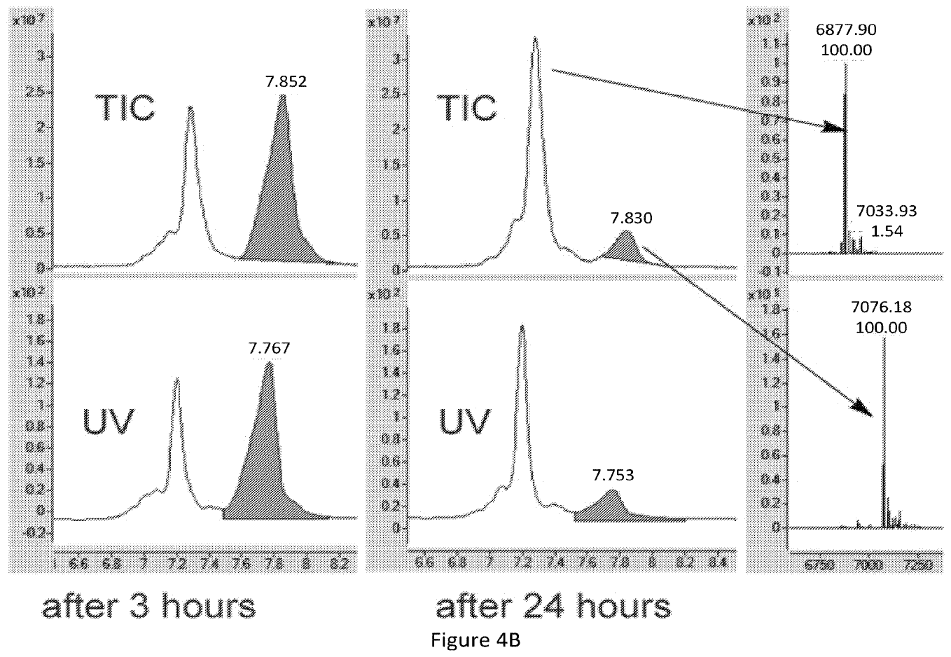

Next, both DNA-conjugated-Fmoc-Asp-derived phosphorane-ylides 4a and 4b were Fmoc deprotected (10% aqueous piperidine) for an hour to produce phosphorane-ylides 5a and 5b (FIG. 3). The deprotected phosphorane-ylides 5a and 5b were observed as expected. However, the hydrolyzed ylide product was also detected in deprotected 5b. Although Fmoc removal was completed in an hour, it was next investigated how long this treatment could be carried out without major hydrolysis of the ylide. Indeed, to generate peptidyl-ylides, 1 hour of deprotection (10% aqueous piperidine) is required for each amino acid addition during peptide elongation. Therefore, the same on-DNA vlide molecule can be exposed to several hours of such treatment. To evaluate the tolerance of such ylides toward hydrolysis, piperidine treatment of both phosphorane-ylides 4a and 4b was performed for up to 24 hours. DNA-conjugated compound 5a was stable with minimal hydrolysis observed by LC-MS analysis even after 24 hours of treatment (FIG. 4). Compound 5b, however, was 50% hydrolyzed after 3 hours of treatment and >90% of the vlide was hydrolyzed after 24 hours (FIG. 4) (Ahsanullah, et al., 2012, Organic Letters, 14(1):14-17; Byrne, P. A., et al., 2016, Chemistry, 22(27):9140-9154). Ylide 5a was then capped with benzoic acid through amidation and ylide 6 (FIG. 3) was obtained.

### Optimization of Wittig Olefination Conditions

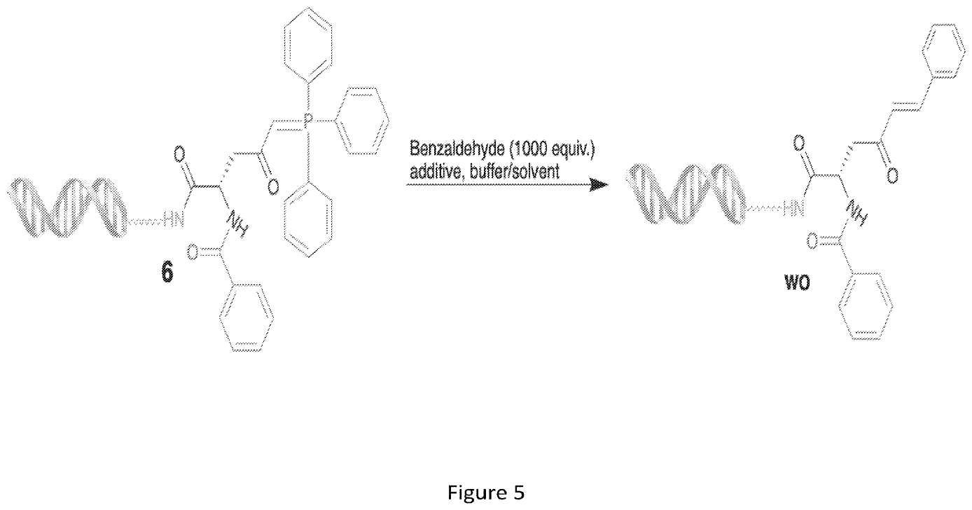

It was next determined the most suitable reaction condition for Wittig olefination to obtain α,β-unsaturated ketone using Asp-derived ylide 6 and benzaldehyde as the model substrate (FIG. 5). Initially, the reaction was carried out in borate buffer pH 9.5 and acetonitrile as co-solvent in presence of DIPEA as base. Benzaldehyde and DIPEA were first used at 100 equivalents at 60° C. for 8 hours and the desired product was observed with a 26% yield (Table 1, Entry 1). The equivalents of both the aldehyde and DIPEA were then increased progressively up to 1000 equivalents. The yield of the desired product was maximized (60%) at the highest equivalents (Table 1, Entry 4). It was next investigated if different inorganic bases such as Na₂CO₃, Cs₂CO₃ and NaOH, influence the conversion rate (Table 1, Entries 5, 6, 9, and 10). None of the inorganic bases lead to improved yields, and the strongest base (NaOH) lead to the highest ylide hydrolysis observed. High amounts of hydrolysis were also observed with acidic additives such as KH₂PO₄ and benzoic acid (Table 1, Entries 7, 8, 11, and 12). Milder acidic conditions (phosphate buffer pH 6.5) did not significantly improve the yield. These results indicate that strong bases and acidic conditions are similarly hampering the reaction by increasing ylide hydrolysis. Selecting DIPEA as a suitable base, DMSO and DMA were tested as alternate co-solvents (Table 1, Entries 13 and 14), with the desired product observed at very low conversion rates. Different ratios of borate buffer (pH 9.5) to acetonitrile were shown to influence the reaction, with increased conversion rates with higher ratios of aqueous medium/borate buffer. Similarly, reducing the temperature from 60° C. to 50° C. also improved the conversion rate (Table 1, Entries 23-26). The optimal conditions were ultimately found to be water as a co-solvent, the aldehyde at 1,000 equiv., DIPEA as the base at 1,000 equiv., and incubation at 50° C. for 12 hours (Table 1, Entry 23).

In these optimized conditions, a single LC-MS peak corresponding to the on-DNA olefination product was observed, indicating that only the E-isomer olefin was formed. However, the presence of a minor quantity of the Z-isomer cannot be excluded. Although isomers typically are not identified by LC-MS, in some cases, depending on their polarity, identification is possible (An, Y. L., et al., 2020, Organic Letters, 22(10):3931-3935).

TABLE 1
Optimization of Wittig reaction conditions.

| | | | Temperature | Conversion |
|---|---|---|---|---|
| Entry | Buffer/Solvent (Ratio) | Additive (equiv.) | (° C.)/Time (h) | (%) |
| 1 | Borate pH 9.5/CH₃CN (1:1) | DIPEA (100)^{a} | 60/8 | 26 |
| 2 | Borate pH 9.5/CH₃CN (1:1) | DIPEA (200)^{b} | 60/8 | 34 |
| 3 | Borate pH 9.5/CH₃CN (1:1) | DIPEA (300)^{c} | 60/8 | 51 |
| 4 | Borate pH 9.5/CH₃CN (1:1) | DIPEA (1000) | 60/8 | 60 |
| 5 | Borate pH 9.5/CH₃CN (1:1) | Na₂CO₃ (1000) | 60/8 | 39 |
| 6 | Borate pH 9.5/CH₃CN (1:1) | Cs₂CO₃ (1000) | 60/8 | 41 |
| 7 | Borate pH 9.5/CH₃CN (1:1) | PhCOOH (100) | 60/8 | 15 |
| 8 | Borate pH 9.5/CH₃CN (1:1) | PhCOOH (1000) | 60/8 | 0 |
| 9 | Borate pH 9.5/CH₃CN (1:1) | NaOH (100) | 60/8 | 30 |
| 10 | Borate pH 9.5/CH₃CN (1:1) | NaOH (1000) | 60/8 | 17 |
| 11 | Borate pH 9.5/CH₃CN (1:1) | KH₂PO₄ (100) | 60/8 | 21 |
| 12 | Borate pH 9.5/CH₃CN (1:1) | KH₂PO₄ (1000) | 60/8 | 0 |
| 13 | Borate pH 9.5/DMSO (1:1) | DIPEA (1000) | 60/8 | 13 |
| 14 | Borate pH 9.5/DMA (1:1) | DIPEA (1000) | 60/8 | 0 |
| 15 | Borate pH 9.5/CH₃CN (1:3) | DIPEA (1000) | 60/8 | 30 |
| 16 | Borate pH 9.5/CH₃CN (3:1) | DIPEA (1000) | 60/8 | 60 |
| 17 | H₂O/CH₃CN (1:1) | DIPEA (1000) | 60/8 | 65 |
| 18 | H₂O/CH₃CN (1:3) | DIPEA (1000) | 60/8 | 32 |
| 19 | H₂O/CH₃CN (3:1) | DIPEA (1000) | 60/8 | 62 |
| 20 | Borate pH 8.5/CH₃CN (1:1) | DIPEA (1000) | 60/8 | 34 |
| 21 | Borate pH 8.5/CH₃CN (3:1) | DIPEA (1000) | 60/8 | 38 |
| 22 | Phosphate pH 6.5/CH₃CN (1:1) | DIPEA (1000) | 60/8 | 30 |
| 23 | H₂O/CH₃CN (3:1) | DIPEA (1000) | 50/12 | 73 |
| 24 | H₂O/CH₃CN (3:1) | DIPEA (500)^{c} | 50/12 | 48 |
| 25 | H₂O/CH₃CN (1:1) | DIPEA (1000) | 50/12 | 67 |
| 26 | H₂O/CH₃CN (3:1) | DIPEA (1000) | 50/12 | 65 |
| 27 | H₂O/CH₃CN (1:1) | DIPEA (1000) | 60/12 | 62 |

Conditions: PhCHO (1000 equiv.);
Buffer/Solvent volume (20 μL);
^{a}PhCHO (100 equiv.);
^{b}PhCHO (200 equiv.);
^{c}PhCHO (500 equiv.);
optimum results bolded.

### Examination of Aldehyde Substrate Scope

Once the optimal reaction conditions for the Wittig olefination were determined, the substrate scope of the reaction was examined. As illustrated in FIG. 6, a total of 47 aldehydes were tested with Asp-derived ylide 6. The desired products were obtained with appreciable yields for the three categories of aldehydes tested: 1) substituted aromatic aldehydes, 2) heteroaryl aldehydes with the aldehyde at a variety of positions, and 3) aliphatic aldehydes. Aromatic aldehydes with halogen substituents (e.g., fluorine, chlorine, bromine, and iodine) lead to their corresponding products with yields ranging from 56% o to 87% o regardless of para-, meta- or ortho-position (W1-W3, W12, W22). Electron-rich substituents (e.g., methoxy, acetamido, methyl, and phenyl) at the para position of aryl aldehydes gave their corresponding products in a range of acceptable yields (W4-W6, W8). Para-substituted acetoxy benzaldehyde resulted in the expected product with ayvield of 820%, although 3000 of the product had deacetylated (W9). Notably, both alkyne- and alkene-substituted aryl aldehydes generated the desired products in yields of 61%-86% (W10, W11, W21). Functional groups such as ketone and ester were well-tolerated, and the corresponding products were obtained with similar conversion rates without ester hydrolysis (W15-16). Surprisingly, electron-rich compounds meta-substituted or sterically hindered ortho-substituted aldehydes gave the corresponding α,β-unsaturated ketones in substantial yields (W13, W23). Aldehyde derivatives with electron-withdrawing substituents such as trifluoromethyl, nitrile, methyl sulfonyl, and nitro afforded the desired products with yields ranging from 68%-86% (W17-20). However, some aldehyde-DNA adducts were observed with the strong electron-withdrawing aldehyde derivatives. Di-substituted electron-rich aryl aldehyde derivatives and naphthaldehyde derivatives reacted with the ylide and furnished the desired products with appreciable yields (W25-28, W29-30). Heteroaryl aldehydes such as 2-pyridinecarboxaldehyde. 3-pyridine-carboxaldehyde, furaldehyde, thiophene-3-carboxaldehyde, benzofuran-3-carboxaldehyde, 6-quinolinecarbaldehyde, 1,3-benzothiazole-2-carbaldehvde and oxazole-4-carboxaldehyde provided the desired α,β-unsaturated compounds with significant yields (W32-40). Indole-carboxaldehyde and pyrazole-carboxaldehyde resulted in lesser conversion though aliphatic aldehydes were well tolerated (W41-46).

### On-DNA Peptide Synthesis

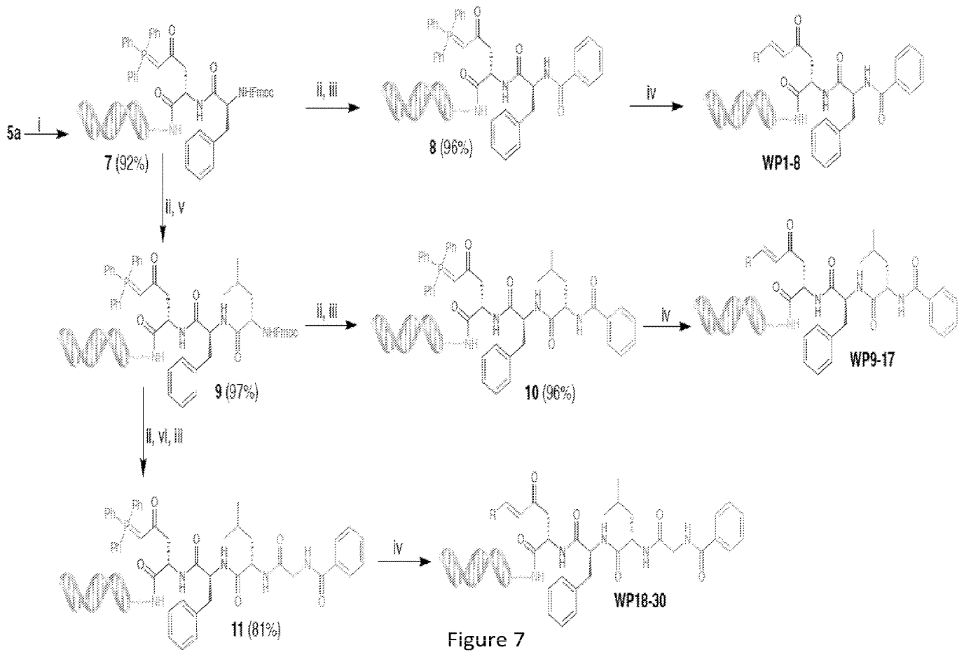

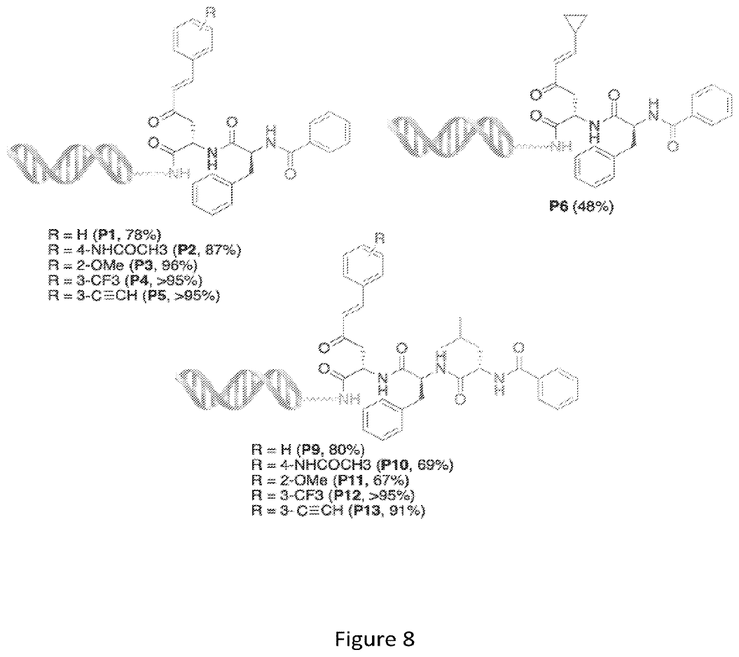

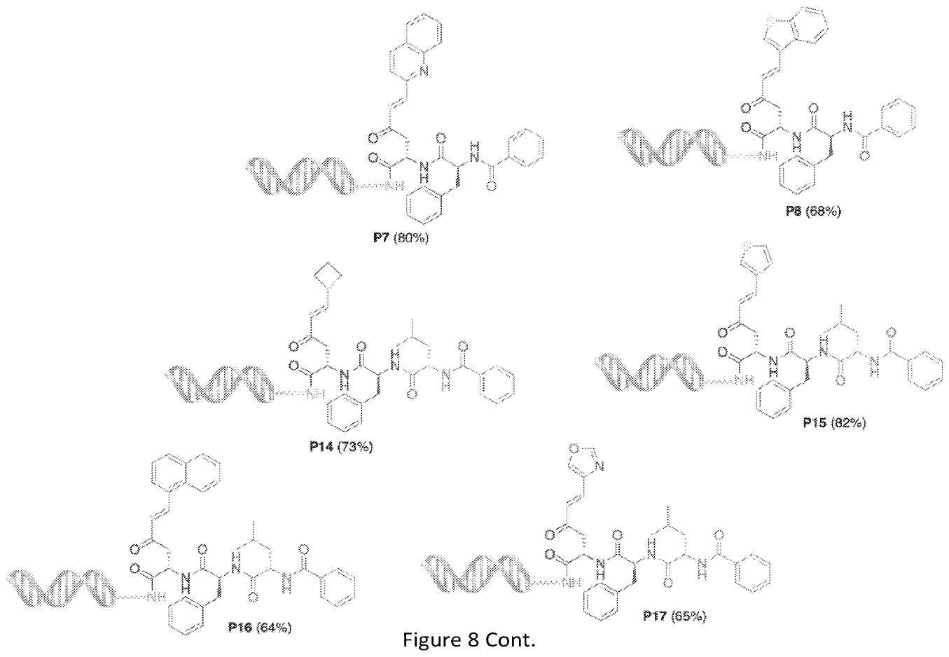

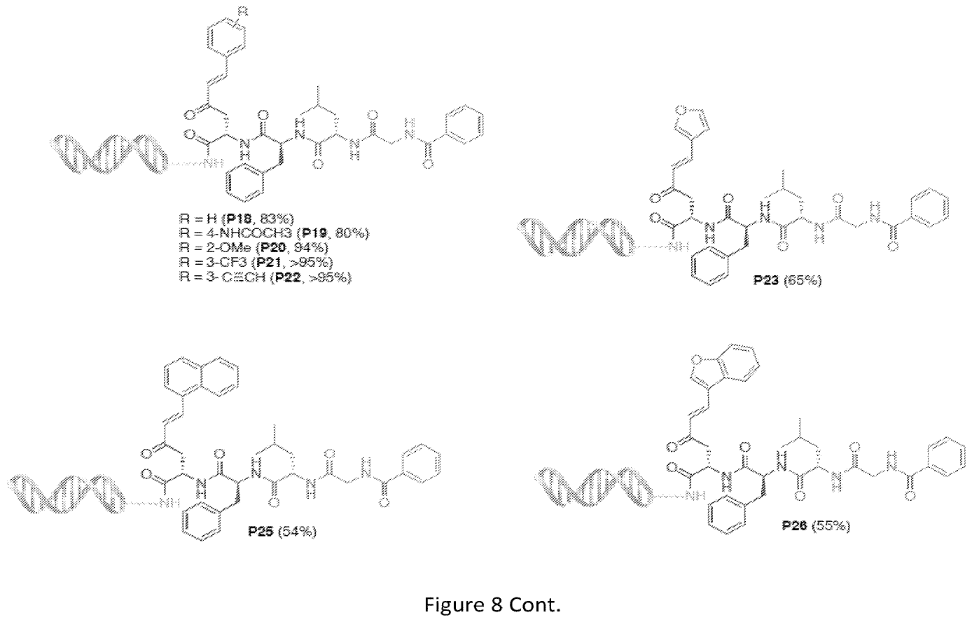

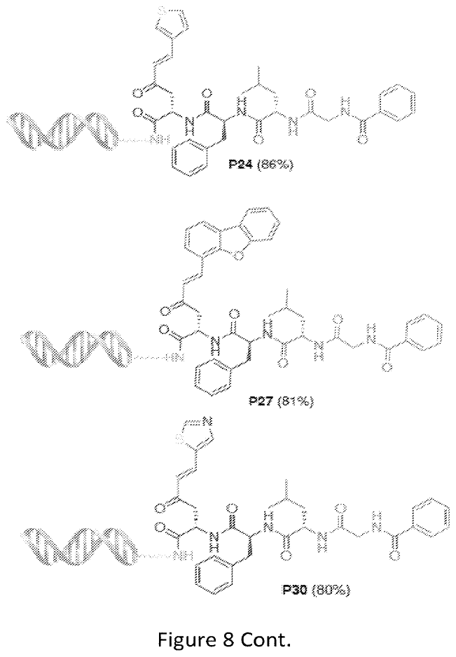

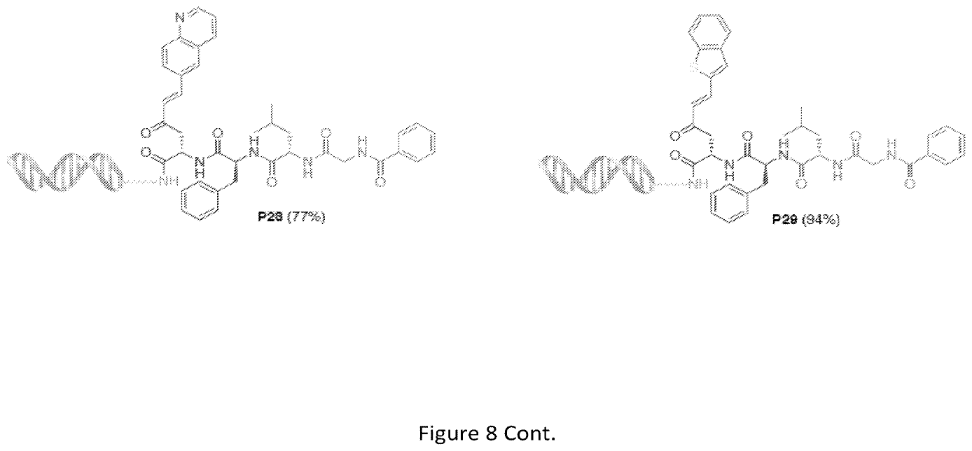

Encouraged by the broad substrate scope for Wittig olefination of a monomeric Asp-based ylide, the conditions for synthesizing on-DNA peptidyl-ylides were investigated. To synthesize on-DNA peptidyl-ylides, monomeric ylide 5a was coupled with Fmoc-Phe-OH using EDC/HOAt/DIPEA in MOPS (pH 8.0, 0.5M NaCl), providing dipeptidyl-ylide 7 with a 92% yield. Fmoc removal and subsequent amine capping of compound 7 lead to on-DNA dipeptidyl-ylide 8 with 96% yield (FIG. 7). Dipeptidyl-ylide 8 was then used with a variety of aldehydes to provide the corresponding α,β-unsaturated dipeptidyl ketones. The reaction with benzaldehyde provided peptide P1 presented in FIG. 8 with 78% yield. Notably, aldehydes bearing electron-rich substituents at the 2- or 4-position lead to P2 and P3 with yields of 87% and 96% respectively. Reaction with an aldehyde bearing a strong electron withdrawing group yielded P4 with an >95% yield. Substitution of a benzaldehyde for an aliphatic aldehyde led to P6 with a 48% yield. Heteroaromatic aldehydes containing oxygen and sulfur also led to the desired products P7 and P8 with yields of 80% and 68% respectively.

Tri- and tetra-peptidvl-ylides were synthetized next (FIG. 7, middle and bottom lines respectively). The Fmoc group of compound 7 was removed, and the resulting amine coupled with Fmoc-Leu-OH to provide the Fmoc-protected-tripeptidyl-ylide 9 with a 97% conversion rate. Compound 9 was then treated with piperidine and coupled with benzoic acid to form tripeptidyl-ylide 10 with a 96% yield. Tetra peptidyl-ylide 11 was achieved with an 81% conversion rate from compound 9 following Fmoc-Gly-OH coupling, piperidine treatment, and coupling to benzoic acid. Tri- and tetra-peptidyl-ylides were subjected to Wittig olefination with 9 and 13 different aldehydes, respectively. These aldehydes presented different steric and electronic substituents, including aromatic, heteroaromatic and aliphatic substituents. Aldehydes with both electron-rich and electron-withdrawing substituents at ortho-, meta- and para-positions all delivered the desired α,β-unsaturated peptidyl ketones with yields for some aldehydes approaching near-complete conversion (P9-13, tripeptidyl vinyl ketones; P18-22, tetra peptidyl vinyl ketones) (FIG. 8). An aliphatic aldehyde and naphthaldehyde reacted with peptidyl-ylides to afford the desired products in moderate yields (P14, P16 and P25). Heteroaryl aldehydes were well tolerated in on-DNA reaction conditions leading to the desired α,β-unsaturated peptidyl ketones (P15, P17, P23-24, P26-30) with near complete conversion in some instances (FIG. 8). Specific side products were observed for a few aldehydes used, corresponding to specific Wittig ylide products.

### Implementation of Diversity-Oriented Synthesis (DOS)

After successful synthesis of various α,β-unsaturated ketones ranging from a single amino acid to tetra-peptides, diversity-oriented synthesis (DOS) strategy was applied to the peptidyl vinyl ketones (FIG. 11). Indeed, α,β-unsaturated ketone moieties are important intermediates for diversification with nucleophiles due to their electron deficient nature. Tetra-peptidyl ketone P18 was treated with hydroxyl amine or hydrazine to afford the isooxazoline and pyrazoline-derived peptides PD1 and PD3 with 77% and 91% conversion rates, respectively. Peptides PD2 and PD4 were obtained similarly with high conversion rates. Dihydropyrimidine-based tripeptide PD5 was obtained with an 86% yield by treating peptidyl vinyl ketone P13 with guanidine in the presence of sodium hydroxide.

Investigation into Glutamic Acid-Based Synthesis

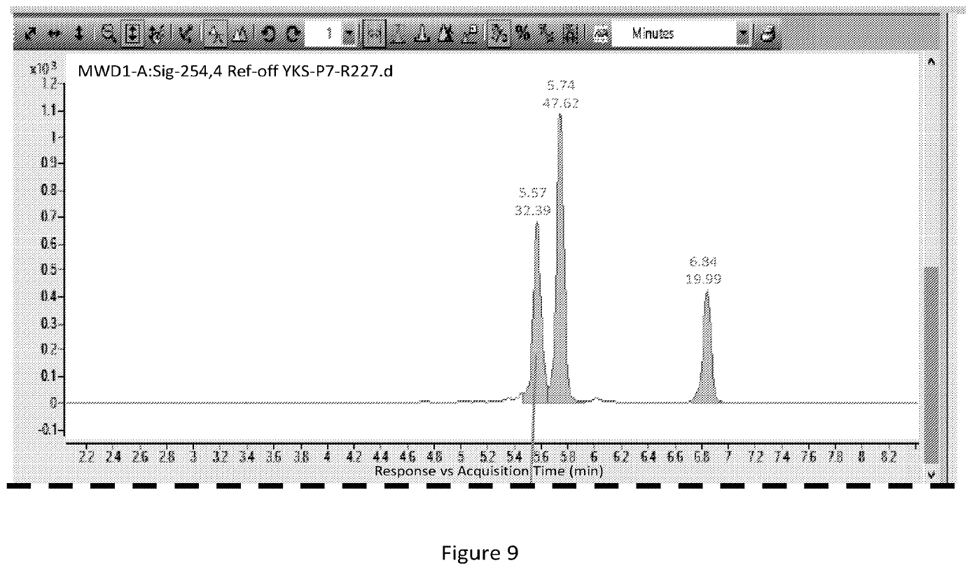

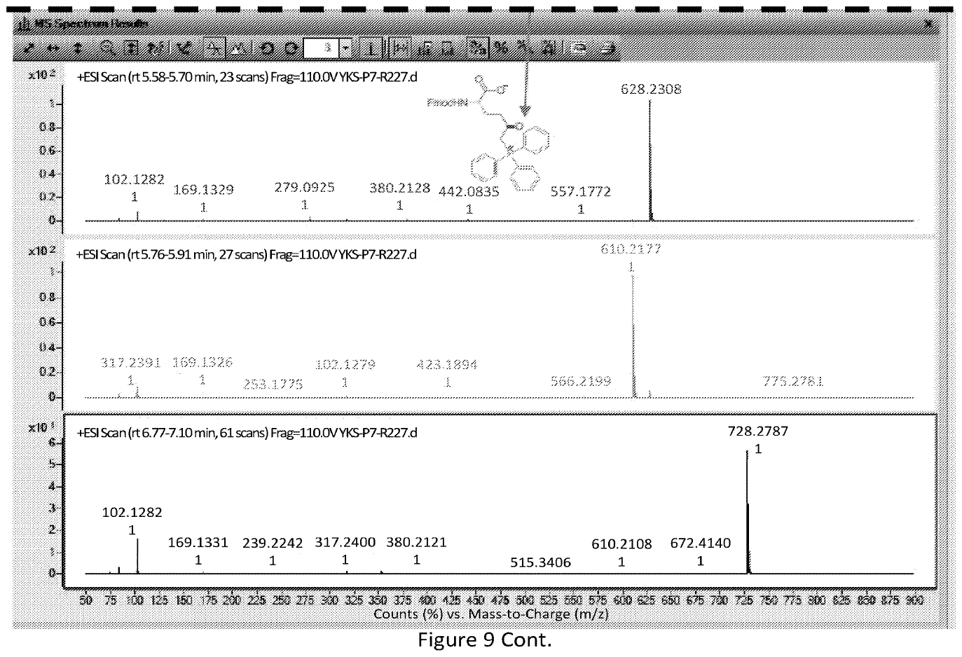

Having successfully developed a synthetic platform with aspartic acid, glutamic acid was next investigated with the same approach. N-Fmoc-L-glutamic acid-α-tert-butyl ester was C-acylated with salt Ta to give phosphorane 12. After cleavage of tert-butyl ester and instantaneous decarboxylation of phosphorane 12, a second major product, 13b, was observed by LC-MS together with the expected Fmoc-Glu-derived phosphonium salt 13a. It was found that 13b corresponds with the cyclized product formed through intramolecular C-acylation as shown in FIG. 9. Furthermore, a longer reaction time led to the full disappearance of the starting material and of desired product 13a. Investigations into the use of glutamic acid are ongoing.

### Aromatic Linkers

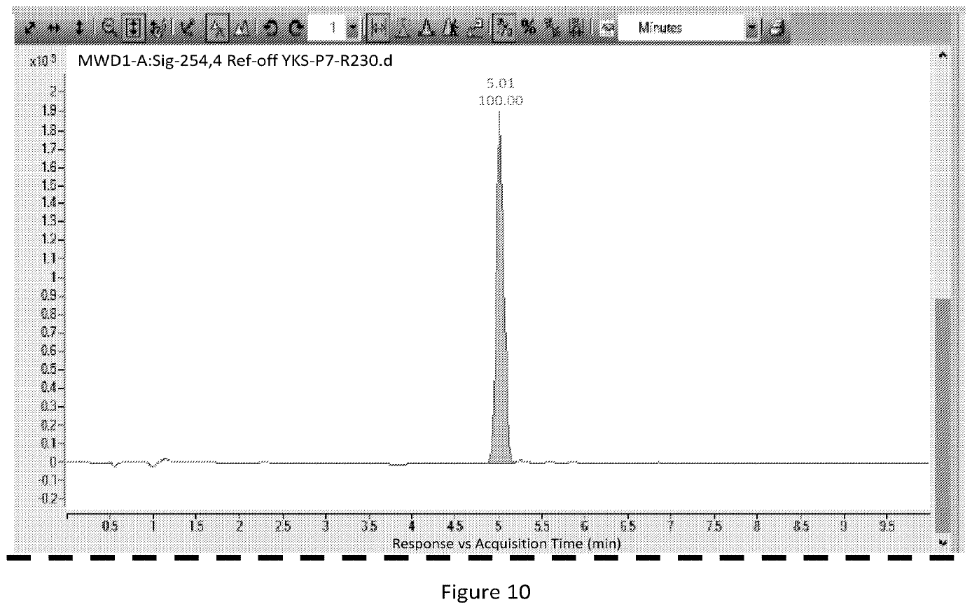

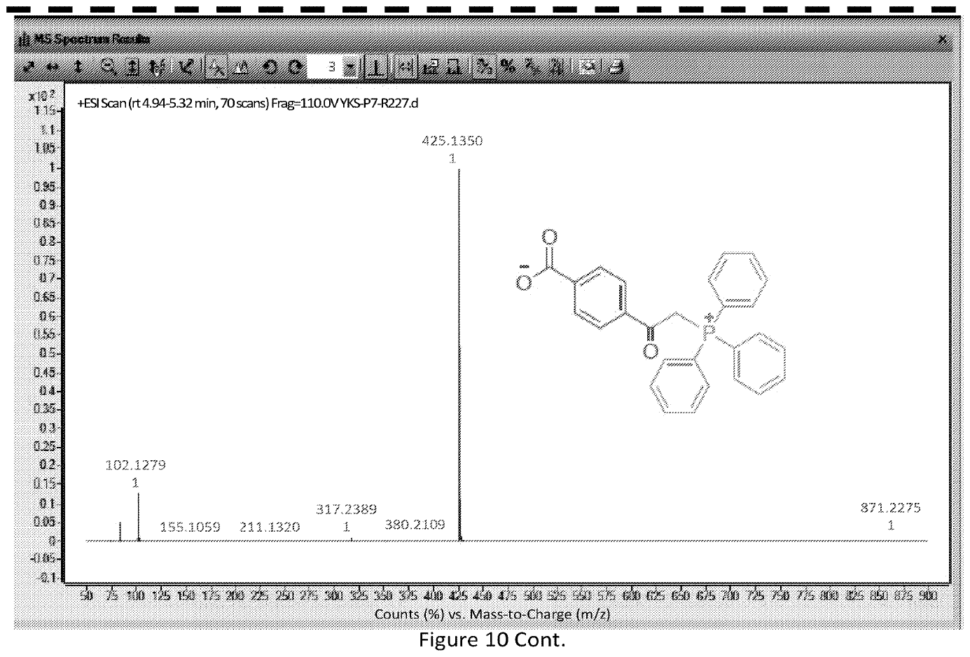

Wittig ylide 3a was derived from aspartic acid and the stabilized ylide was attached to the aliphatic chain. In order to optimize the Wittig olefination condition for aromatic moieties, a stabilized vlide linked to an aromatic moiety was examined. 4-(tert-butoxycarbonyl)benzoic acid was coupled with phosphonium salt 1a and followed by trifluoroacetic acid treatment which led to compound 14. The coupling of compound 14 with DNA1 produced on-DNA aryl Wittig ylide 15 (FIG. 10), which was then subjected to Wittig olefination with 56 different aldehydes (FIG. 12). Interestingly, aryl aldehydes containing electron donating groups (e.g., methyl, phenyl, hydroxy, methoxy and acetamido) led to the corresponding chalcone-derived products independently of their ortho-, meta- or para-positions (FIG. 12, W50-52, W59, W69-73). Substantial conversion rates were obtained with weak electron withdrawing groups such as halogens (W47-49. W58, W68). Para-substituted acetoxy benzaldehyde afforded the expected product with 82% yield, although 30% was deacetylated (W55). Remarkably, functional groups (e.g., carboxylic acid, ketone, ester, cyano, and nitro groups) were well-tolerated and led to the corresponding chalcone-derivatives in excellent yields (W60-62, W64 & W66). Doubly-substituted aryl aldehydes and naphthalene aldehydes gave the desired products in with conversion rates ranging from 66% to 92% (W74-79). Heteroaryl aldehydes containing mono- to tri-cyclic aromatic rings provided the corresponding chalcone derivatives with over 95% conversion in most cases (W81-99). Aliphatic aldehydes were well-tolerated and gave the corresponding α,β-unsaturated ketones in appreciable yields (W100-102).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

The methods and materials employed are described herein.

### Reagents

All chemical reagents were purchased from commercial sources and used without further purification, unless otherwise noted. DNA1 was custom designed in-house and synthesized by Integrated DNA Technologies, Inc. Lyophilized DNA was resuspended in T.E. pH 7.4 and used without further purification.

### LC-MS Instrumentation, Acquisition Conditions, and Data Analysis:

Agilent Instruments: LC-MS analyses were performed using an Agilent LC-MS system (LC-MS-TOF 6230B, Agilent, Santa Clara, CA, USA) according to the manufacturer instructions. LC components included a multisampler (model number G7167A), binary pump (model number G7112B), column compartment (model number G7116A), UV/MWD detector (model number G7165A), and MS TOF (model number G6230B).

Analysis Conditions: The mobile phase consisted of 100 mM hexafluoro-2-propanol (HFIP)/8.9 mM triethylamine (TEA) in deionized water (A) and methanol (MeOH) (B). The samples were injected onto an RP chromatography column (Targa C18, 5 μm, 50×2.1 mm, 120 Å) and subjected to gradient elution (1% B for 1 minute, 1%-70% B for 12 minutes, and equilibration for 3 minutes) at a flow rate of 0.4 mL/min with a column temperature of 40° C. Dual ESI negative mode polarity was used with a scan range of 500-3200 Da. The source conditions were as follows: drying gas flow 12 L/min at 325° C. with a nebulizer pressure of 30 psi. The capillary voltage was set to 4,000 V.

Data Acquisition and Analysis: The data for each DNA sample were acquired using the software “MassHunter Workstation Data Acquisition” (Agilent) and the data were analyzed using “MassHunter Qualitative Analysis B.07.00” (Agilent). The estimated yields of DNA samples were determined by examination of the appropriate peaks using both the UV absorbance traces at 260 nm and Total Ion Chromatogram (TIC) traces.

### Preparation of Triphenylphosphonium Bromide Salt 1a

To a stirring solution of triphenylphosphine (1.23 g, 4.71 mmol) in dry CH₂Cl₂, tert-butyl-2-bromoacetate (1 equiv., 695 μL, 4.71 mmol) was added and the reaction was stirred at room temperature for 20 hours. The solvent was removed under vacuum to afford a white color solid (97% yield).

Preparation of diphenylmethylphosphonium bromide salt 1b

To a stirring solution of diphenylmethylphosphine (309 μL, 2.17 mmol) in dry CH₂Cl₂, tert-butyl-2-bromoacetate (1 equiv., 321 μL, 2.17 mmol) was added and the reaction was stirred at room temperature for 20 hours. The solvent was removed under vacuum to afford a white color solid (81% yield).

### Synthesis of Compound 2a

To a stirring solution of Fmoc-Asp-OtBu (110 mg, 0.27 mmol) in dry CH₂Cl₂, compound 1a (1 equiv., 122 mg, 0.27 mmol) was added. BTFFH (1 equiv., 84 mg, 0.27 mmol) was added to the reaction mixture and followed by addition of DIPEA (5 equiv., 232 μL, 1.34 mmol). The reaction mixture was then stirred at room temperature overnight. The solvent was removed under vacuum and the crude product was purified by using flash chromatography to afford compound 2a (79% yield).

### Synthesis of Compound 2b

To a stirring solution of Fmoc-Asp-OtBu (110 mg, 0.27 mmol) in dry CH₂Cl₂, compound 1b (1 equiv., 105 mg, 0.27 mmol) was added. BTFFH (1 equiv., 84 mg, 0.27 mmol) was added to the reaction mixture and followed by addition of DIPEA (5 equiv., 232 μL, 1.34 mmol). The reaction mixture was then stirred at room temperature overnight. The solvent was removed under vacuum and the crude product was purified by using flash chromatography to afford compound 2b (76% yield).

### Synthesis of Compound 3a

To a stirring solution of compound 2a in dry CH₂Cl₂, TFA was added and stirred at room temperature for 4 hours. The solvent was removed under vacuum and the crude product was purified by using flash chromatography to afford compound 3a (89% yield).

### Synthesis of Compound 3b

To a stirring solution of compound 2b in dry CH₂Cl₂, TFA was added and stirred at room temperature for 4 hours. The solvent was removed under vacuum and the crude product was purified by using flash chromatography to afford compound 3b (88% yield).

### Preparation of DNA2 by linker attachment to DNA1 (DNA2)

To DNA1 (120 nmol, 1 mM in MOPS pH 8.0, 0.5 M NaCl), Fmoc-NH-PEG4-COOH, EDC, HOAt, and DIPEA (60 μL each, 100 equiv., 200 mM DMSO) were added and the reaction mixture was stirred at 25° C. for 4 hours. Precipitated DNA was resuspended in H₂O (120 μL). 120 μL of 20% piperidine/H₂O was added and the reaction mixture was stirred at 25° C. for 1 hour. The resuspended DNA was quantified using NanodropOne under dsDNA mode.

### Conjugation of 3a to DNA2 (4a)

To DNA2 (100 nmol, 1 mM in MOPS pH 8.0, 0.5 M NaCl), compound 3a (10 μL, 20 equiv., 200 mM DMSO), EDC, HOAt, and DIPEA (50 μL each, 100 equiv., 200 mM DMSO) were added and the reaction mixture was stirred at 25° C. for 4 hours. 10 μL of 5 M NaCl and 1 mL EtOH were added, and the reaction kept at −80° C. for 1 hour. The colloidal solution was centrifuged for 30 minutes at 4° C. at 13,000 rpm and the supernatant was carefully removed. The pellet was washed once, dried, and resuspended in MOPS pH 8.0, 0.5 M NaCl (100 μL). The resuspended DNA 4a was quantified using NanodropOne under dsDNA mode.

### Conjugation of 3b to DNA2 (4b)

To DNA2 (10 nmol, 1 mM in MOPS pH 8.0, 0.5 M NaCl), compound 3b (1 μL, 20 equiv., 200 mM DMSO), EDC, HOAt, and DIPEA (5 μL each, 100 equiv., 200 mM DMSO) were added and the reaction mixture was stirred at 25° C. for 4 hours. 2 μL of 5 M NaCl and 1 mL EtOH were added, and the mixture kept at −80° C. for 1 hour. The colloidal solution was centrifuged for 30 minutes at 4° C. at 13,000 rpm and the supernatant was carefully removed. The pellet was washed once, dried, and resuspended in H₂O (10 μL).

### Deprotection of 4a (5a)

100 μL of 20% piperidine/H₂O was added to 4a (100 nmol, 100 μL, 1 mM in H₂O) and the reaction mixture was stirred at 25° C. for 24 hours. 10 μL of 5 M NaCl and 1 mL EtOH were added, and the mixture kept at −80° C. for 1 hour. The colloidal solution was centrifuged for 30 minutes at 4° C. at 13,000 rpm and the supernatant was carefully removed. The pellet was washed once, dried, and resuspended in MOPS pH 8.0, 0.5 M NaCl, (100 μL).

### Deprotection of 4b (5b)

10 μL of 20% piperidine/H₂O was added to 4a (10 nmol, 10 μL, 1 mM in H₂O) and the reaction mixture was stirred at 25° C. for 24 hours. 10 μL of 5 M NaCl and 1 mL EtOH were added, and the mixture kept at −80° C. for 1 hour. The colloidal solution was centrifuged for 30 minutes at 4° C. at 13.000 rpm and the supernatant was carefully removed. The pellet was washed once, dried, and resuspended in MOPS pH 8.0, 0.5 M NaCl, (10 μL).

### Synthesis of Compound 6

To 5a (50 nmol, 50 μL, 1 mM in MOPS pH 8.0, 0.5 M NaCl), benzoic acid, EDC, HOAt, and DIPEA (25 μL each, 100 equiv., 200 mM DMSO) were added, and the reaction mixture was stirred at 25° C. for 4 hours. 10 μL of 5 M NaCl and 1 mL EtOH were added, and the mixture kept at −80° C. for 1 hour. The colloidal solution was centrifuged for 30 minutes at 4° C. at 13,000 rpm and the supernatant was carefully removed. The pellet was washed once, dried, and resuspended in 50 μL of H₂O.

### Synthesis of Peptidyl-ylides 8, 10, and 11

To prepare on-DNA peptidyl-ylides, on-DNA monomeric Asp-derived ylide 5a was elongated. Sequential addition of Fmoc-protected amino acids and the deprotection of Fmoc groups resulted in the corresponding peptidyl-ylides. For preparing compound 8, Fmoc-Phe-OH, EDC, HOAt, and DIPEA (25 μL each, 100 equiv., 200 mM DMSO) were added to 5a (50 nmol, 50 μL. 1 mM in MOPS pH 8.0, 0.5 M NaCl), and the reaction mixture was stirred at 25° C. for 4 hours, resulting in compound 7 in protected form. 10 μL 5 M NaCl and 1 mL EtOH were added, and the mixture kept at −80° C. for 1 hour. The colloidal solution was centrifuged for 30 minutes at 4° C. at 13,000 rpm and the supernatant was carefully removed. The pellet was washed once, dried, and resuspended in 50 μL of H₂O. Fmoc protection was removed from 7 using 10% piperidine in H₂O for 1 hour and the mixture was precipitated as above. 15 μL of compound 7 was coupled with benzoic acid in the presence of EDC, HOAt, and DIPEA to afford dipeptidyl-ylide 8. In a similar way, tripeptidyl-ylide 10 starting from 7 (15 μL) and tetrapeptidyl-ylide 11 starting from 7 (20 μL) were synthesized.

### Synthesis of Compound 12

To a stirring solution of Fmoc-Glu-OtBu (116 mg, 0.27 mmol) in dry CH₂Cl₂, compound 1a (1.1 equiv., 137 mg, 0.3 mmol) was added. BTFFH (1.5 equiv., 126 mg, 0.41 mmol) was added to the reaction mixture and followed by addition of DIPEA (3 equiv., 142 μL, 0.82 mmol). The reaction mixture was then stirred at room temperature overnight. The solvent was removed under vacuum and the crude product was purified by using flash chromatography to afford compound 12 (32% yield).

### Synthesis of Compound 13

To a stirring solution of compound 12 in dry CH₂Cl₂, TFA was added and stirred at room temperature for 4 hours.

### Synthesis of Compound 14

To a stirring solution of 4-(tert-butoxycarbonyl)benzoic acid (104 mg, 0.47 mmol) in dry CH₂Cl₂, compound 1a (1 equiv., 177 mg, 0.47 mmol) was added. BTFFH (1 equiv., 146 mg, 0.47 mmol) was added to the reaction mixture and followed by addition of DIPEA (5 equiv., 406 μL, 2.35 mmol). The reaction mixture was then stirred at room temperature overnight. The solvent was removed under vacuum and the crude product was purified by using flash chromatography to afford the acid-protected compound. TFA was added to the acid-protected compound in dry CH₂Cl₂ and stirred at room temperature for 4 hours. The solvent was removed under vacuum and the crude product was purified using flash chromatography to afford compound 14 (81% yield).

### Synthesis of Compound 15

To DNA1 (100 nmol, 100 μL, 1 mM in MOPS pH 8.0, 0.5 M NaCl), compound 14 (10 μL, 20 equiv., 200 mM DMSO), EDC, HOAt, and DIPEA (50 μL each, 100 equiv., 200 mM DMSO) were added and the reaction mixture stirred at 25° C. for 4 hours. 10 μL of 5 M NaCl and 1 mL EtOH were added, and the mixture kept at −80° C. for 1 hour. The colloidal solution was centrifuged for 30 minutes at 4° C. at 13,000 rpm and the supernatant was carefully removed. The pellet was washed once, dried, and resuspended in MOPS pH 8.0, 0.5 M NaCl (100 μL).

### General Procedure for Wittig Olefination (WO, W1-102, P1-P30)

1 μL ylide (6/8/10/11/15) (1 mM H₂O) was added to 14 μL H₂O. 2.5 μL of aldehyde and DIPEA were added (400 mM CH₃CN, 1000 equiv.) and the reaction mixture was stirred at 50° C. for 12 hours. 2 μL of 5 M NaCl and 400 μL EtOH were added, and the mixture kept at −80° C., or 1 hour. The colloidal solution was centrifuged for 30 minutes at 4° C. at 13,000 rpm and the supernatant was carefully removed. The pellet was washed once, dried, and resuspended in 20 μL of H₂O. 5 μL was added to 5 μL H₂O for LC-MS analysis; 5 μL was injected on LC-MS.

### Synthesis of Compounds PD1 and PD2

10 μL of α,β-unsaturated peptide ketone (P18/20) (0.1 mM H₂O) was added to 15 μL of phosphate buffer pH 5.5, 2 μL of NH₂OH·HCl (200 mM H₂O, 400 equiv.) was added and the reaction mixture was stirred at 60° C. for 2 hours. 5 μL was directly injected on LCMS.

### Synthesis of Compounds PD3 and PD4

10 μL of α,β-unsaturated peptide ketone (P18/24) (0.1 mM H₂O) was added to 15 μL of borate buffer pH 9.5, 2 μL of NH₂NH₂ HCl (200 mM H₂O, 400 equiv.) was added and the reaction mixture was stirred at 60° C. for 2 hours. 5 μL was directly injected on LCMS.

### Synthesis of Compound PD5

5 μL of α,β-unsaturated peptide ketone (P13) (0.1 mM H₂O) was added to 5 μL of borate buffer pH 9.5, 2.5 μL of guanidine hydrochloride (200 mM H₂O, 1000 equiv.) and 2 μL of NaOH (200 mM H₂O, 800 equiv.) were added and the reaction mixture was stirred at 70° C. for 3 hours. 5 μL was directly injected on LCMS.

### Example 2: On-DNA Generation of α,β-unsaturated Ketones through Horner-Wadsworth-Emmons Reactions

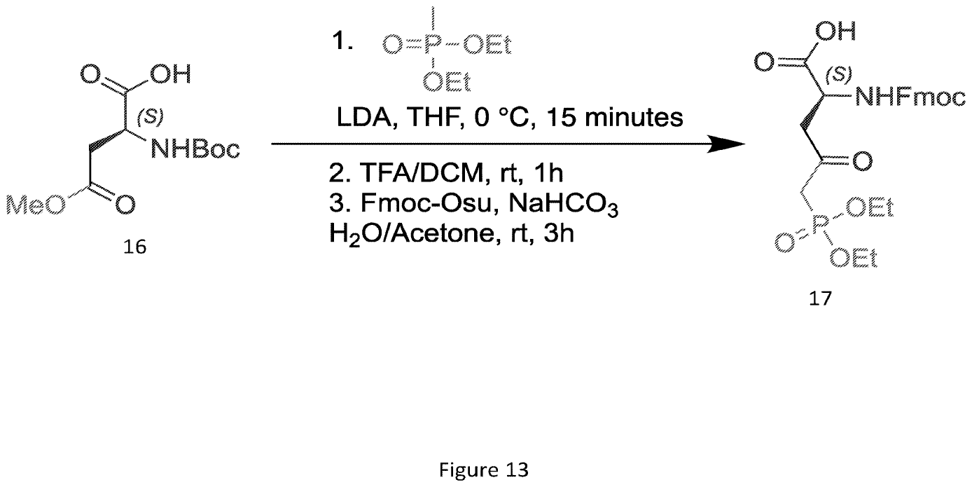

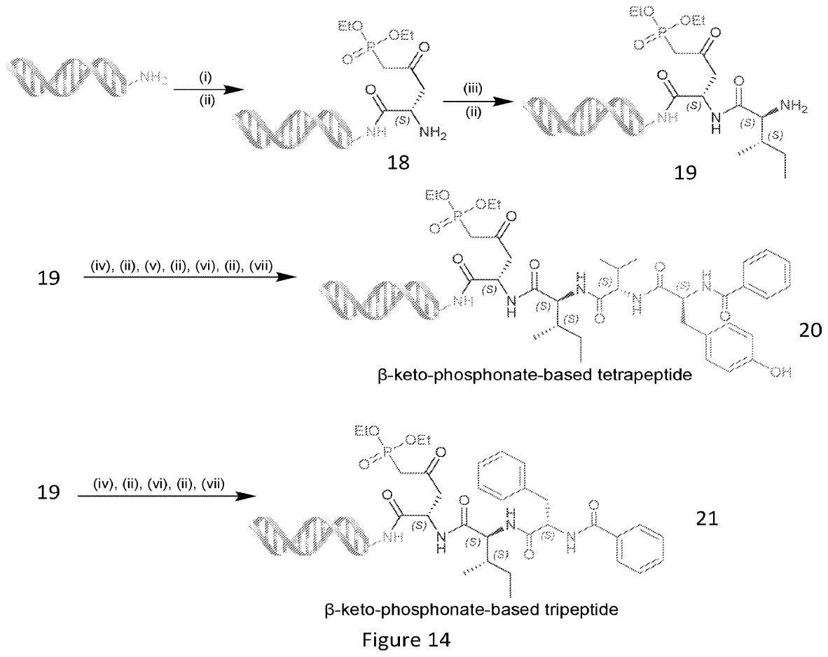

In view of the successful development of a method for generating on-DNA α,β-unsaturated ketones through Wittig reaction from peptidyl-ylides, as an extension of this method was investigated. Another phosphorus-based group, called a β-keto phosphonate, is formed on the side chain of aspartic acid for generating on-DNA α,β-unsaturated ketones through a Wittig-related reaction called the Horner-Wordsworth-Emmons (HWE) reaction. Thus the β-keto phosphonate amino acid was generated starting from aspartic acid (FIG. 13). The β-keto phosphonate amino acids were then conjugated to DNA (FIG. 14). The DNA conjugated-β-keto phosphonate-based amino acid was further extended to create DNA-conjugated tri- and tetrapeptide-based-β-keto phosphonates by addition of sequential amino acids (FIG. 14).

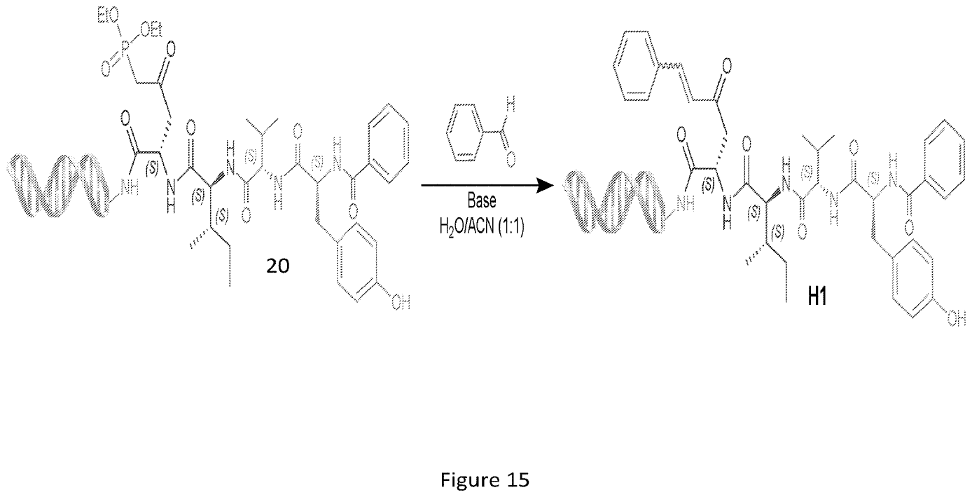

Once DNA-conjugated peptide-based- β-keto phosphonates were prepared, the Homer-Wordsworth-Emmons (HWE) reaction was tested between DNA conjugated tetrapeptide-based- β-keto phosphonate and benzaldehyde, in order to determine the optimal condition for generating on-DNA α,β-unsaturated ketones (FIG. 15, Table 2).

TABLE 2
Conditions optimization for the HWE reaction between DNA conjugated tetrapeptide-based-
β-keto phosphonate and benzaldehyde. Reaction conditions: Phosphonate (22, 1 mM in
H₂O, 1 μL), benzaldehyde (200 mM, 2.5 μL for 500 eq, 5 μL for 1,000 eq), KOH (100
mM, 2 μL for 200 eq; 200 mM, 2.5 μL for 500 eq), 50% H₂O/acetonitrile (20 μL).

| Entry | Solvent | Benzaldehyde (eq) | Base (eq) | T(° C.)/Time | Conversion |
|---|---|---|---|---|---|
| 1 | H₂O/CH₃CN (1:1) | 500 | K₂CO₃ (500) | 60/8 h | 50% |
| 2 | H₂O/CH₃CN (1:1) | 1000 | K₂CO₃ (1000) | 60/8 h | 80% |
| 3 | H₂O/CH₃CN (1:1) | 500 | KOH (200) | 60/4 h | 50% |
| 4 | H₂O/CH₃CN (1:1) | 500 | KOH (500) | 60/4 h | 65% |
| 5 | H₂O/CH₃CN (1:1) | 500 | KOH (200) | 40/8 h | 88% |
| 6 | H₂O/CH₃CN (1:1) | 1000 | KOH (200) | 40/8 h | 66% |
| 7 | H₂O/CH₃CN (1:1) | 1000 | KOH (500) | 40/8 h | 92% |
| 8 | H₂O/CH₃CN (1:1) | 500 | KOH (500) | 40/8 h | 85% |

Following optimization, three conditions were used for exploring a substrate scope based on nature of the aldehydes (FIG. 16 and FIG. 17). Various aromatic aldehydes containing different substituents (e.g., halo, donating, and withdrawing groups) at different positions (e.g., ortho, meta, and para) were tested with DNA conjugated tetra and tripeptide-based-β-keto phosphonates. The aldehydes tested produced the corresponding on-DNA α,β-unsaturated ketones in good to excellent yields (FIG. 16 and FIG. 17). Furthermore, the reaction was tested with heteroaryl aldehydes with both DNA-conjugated tetra and tripeptide-based-β-keto phosphonates and all gave the corresponding desired products in almost excellent yields. Notably, aliphatic aldehydes gave good to excellent yields which were even better than with our previous Wittig ylides or any other method for generating on-DNA α,β-unsaturated ketones (FIG. 16 and FIG. 17).

The methods and materials employed are described herein.

### LCMS Instrumentation:

LCMS analyses were performed using an Agilent LCMS system (LCMS-TOF 6230B) (Agilent, Santa Clara, CA, USA) according to the manufacturer instructions. LC components included a multi-sampler (model number—G7167A), binary pump (model number—G7112B), column compartment (model number—G7116A), UV/MWD detector (model number—G7165A), and MS TOF (model number—G6230B).

### Analysis Conditions:

The mobile phase consisted of 100 mM HFIP/8.9 mM TEA in deionized water (A) and MeOH (B). The samples were injected onto an RP chromatography column (Targa C18, 5 μm, 50×2.1 mm, 120 Å) and subjected to gradient elution (1% B for 1 minute; 1%-70% B for 12 minutes, and equilibration for 3 minutes) at a flow rate of 0.4 mL/min, with a column temperature of 40° C. Dual ESI negative mode polarity was used with a scan range of 500-3,200 Da. The source conditions were as follows: drying gas flow 12 L/min at 325° C. with a nebulizer pressure of 30 psi. The capillary voltage was set to 4,000 V.

### Data Acquisition and Analysis:

The data for each DNA sample were acquired using the software ‘MassHunter Workstation Data Acquisition’ (Agilent) and the data were analyzed using ‘MassHunter Qualitative Analysis B.07.00’ (Agilent).

### Yield Calculation:

The estimated yield of DNA samples was determined by examination of the appropriate peaks using Total Ion Chromatogram (TIC) traces.

### Preparation of Asp-Derived β-Keto Phosphonate Amino Acid (16)

To a stirring solution of diethyl methylphosphonate (796 μL, 5.44 mmol) and Boc-Asp(OMe)-OH (1.03 g, 4.19 mmol) in dry THF, LDA (4 mL, 8.32 mmol) was added dropwise at 0° C. and the reaction mixture was stirred for 15 minutes. The reaction mixture was carefully quenched with 6M HCl to acidify and the solvent was removed under vacuum. The crude mixture was extracted with CH₂Cl₂ the solvent was removed under vacuum. 20% TFA/CH₂Cl₂ was added to the mixture and stirred for 1 hour and the solvent was removed under vacuum. The amine functionality was then protected using Fmoc-OSu in Water/Acetone in presence of NaHCO₃. The solvent was removed under vacuum and the crude was purified using flash column chromatography.

### Conjugation of Asp-Derived β-Keto Phosphonate Amino Acid with DNA (17)

EDC, HOAt and DIPEA (100 μL each, 200 equiv., 200 mM DMSO) were added to the #l-keto phosphonate-based amino acid 16 (100 μL each, 200 equiv., 200 mM DMSO), and the reaction mixture was activated for 3 minutes at 20° C. The activated mixture was added to DNA (100 nmol, 1 mM in MOPS pH 8.0, 0.5M NaCl). 100 μL of MOPS buffer (pH 8.0, 0.5M NaCl) was added to the mixture and the whole mixture was stirred at 20° C. for 2 hours on a ThermoMixer C. 60 μL of 5M NaCl and 3 mL EtOH were added, and the mixture kept at −80° C. for 1 hour. The colloidal solution was centrifuged for 30 minutes at 4° C. at 13,000 rpm and the supernatant was carefully removed. The pellet was washed once, dried and resuspended in H₂O (100 μL). 4% piperidine in H₂O (100 μL) was added to the resuspended DNA-conjugated product (1 mM, 100 μL) and stirred at 20° C. for 3 hours on a ThermoMixer C for removing Fmoc protection. The mixture was precipitated as mentioned above. The DNA pellet was then resuspended in 100 μL of MOPS buffer (pH 8.0, 0.5M NaCl).

### Synthesis of DNA-Conjugated Tetrapeptide-Based β-Keto Phosphonate (20)

EDC, HOAt and DIPEA (50 μL each, 200 equiv., 200 mM DMSO) were added to Fmoc-Ile-OH (50 μL each, 200 equiv., 200 mM DMSO), and the reaction mixture was activated for 3 minutes at 20° C. The activated mixture was added to DNA-conjugated compound 18 (50 nmol, 1 mM in MOPS pH 8.0, 0.5M NaCl). 50 μL of MOPS buffer (pH 8.0, 0.5M NaCl) was added to the mixture and the whole mixture was stirred at 20° C. for 2 hours on a ThermoMixer C. 30 μL of 5M NaCl and 1.5 mL EtOH were added, and the mixture kept at −80° C. for 1 hour. The colloidal solution was centrifuged for 30 minutes at 4° C. at 13,000 rpm and the supernatant was carefully removed. The pellet was washed once, dried and resuspended in H₂₀ (50 μL). 4% piperidine in H₂₀ (50 μL) was added to the resuspended DNA-conjugated product (1 mM, 50 μL) and stirred at 20° C. for 3 hours on a ThermoMixer C for Fmoc deprotection. The mixture was precipitated as mentioned above. The DNA pellet was then resuspended in 50 μL of MOPS buffer (pH 8.0, 0.5M NaCl). The process was repeated with Fmoc-Val-OH, Fmoc-Tyr-OH and finally free amine was capped with benzoic acid to get the DNA-conjugated tetrapeptide-based β-keto phosphonate 20. DNA piece was quantified using NanodropOne (ThermoFisher scientific) under dsDNA mode.

### Synthesis of DNA-Conjugated Tripeptide-Based β-Keto Phosphonate (21)

EDC, HOAt and DIPEA (50 μL each, 200 equiv., 200 mM DMSO) were added to Fmoc-Ile-OH (50 μL each, 200 equiv., 200 mM DMSO), and the reaction mixture was activated for 3 minutes at 20° C. The activated mixture was added to DNA-conjugated compound 3 (50 nmol, 1 mM in MOPS pH 8.0, 0.5M NaCl). 50 μL of MOPS buffer (pH 8.0, 0.5M NaCl) was added to the mixture and the whole mixture was stirred at 20° C. for 2 hours on a ThermoMixer C. 30 μL of 5M NaCl and 1.5 mL EtOH were added, and the mixture kept at −80° C. for 1 hour. The colloidal solution was centrifuged for 30 minutes at 4° C. at 13,000 rpm and the supernatant was carefully removed. The pellet was washed once, dried and resuspended in H₂O (50 μL). 4% piperidine in H₂O (50 μL) was added to the resuspended DNA-conjugated product (1 mM, 50 μL) and stirred at 20° C. for 3 hours on a ThermoMixer C for Fmoc deprotection. The mixture was precipitated as mentioned above. The DNA pellet was then resuspended in 50 μL of MOPS buffer (pH 8.0, 0.5 M NaCl). The process was repeated with Fmoc-Phe-OH and finally free amine was capped with benzoic acid to get the DNA-conjugated tripeptide-based #l-keto phosphonate 21. DNA piece was quantified using NanodropOne (ThermoFisher scientific) under dsDNA mode.

### General Procedure for Homer-Wordsworth-Emmons (HWE) Olefination (H1-H46)

1 μL β-Keto phosphonate-based peptide (1 mM H₂O) was added to 6.5 μL H₂O. 7.5 μL of acetonitrile was added. 2.5 μL of aldehyde (200 mM CH₃CN, 500/1000 equiv.), KOH/K₂CO₃ (200 mM H₂O, 500 equiv.) were added, and the reaction mixture was stirred at 40° C./60° C. for 8 hours on a ThermoMixer C. 6 μL was injected on LCMS.

1. A compound having a structure selected from the group consisting of: 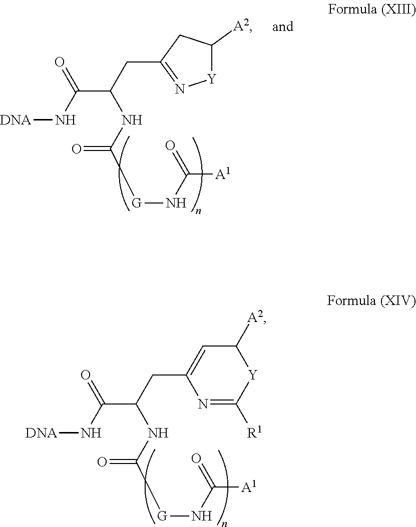
wherein DNA is a nucleic acid barcode;
each instance of G is independently of the formula 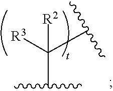
each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R¹, R², and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NR⁴;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

2. The compound of claim 1, wherein the compound has a structure selected from the group consisting of at least one structure as shown in FIG. 11.

3. A method of generating the compound of claim 1:
a) wherein the compound is a compound having the Formula (XIII) and the method comprises:
reacting a compound having the structure of 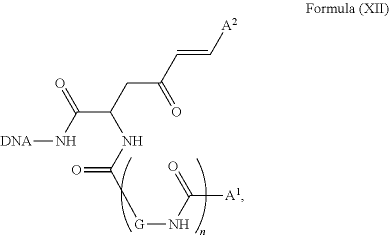
with the compound having the structure of 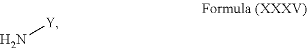
or
b) wherein the compound is a compound having the Formula (XIV) and the method comprises:
reacting the compound having the structure of 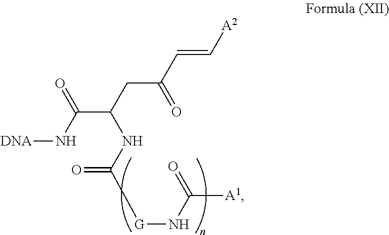
with the compound having the structure of 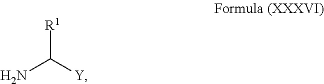
wherein DNA is a nucleic acid barcode;
each instance of G is independently of the formula 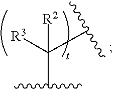
each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R¹, R², and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NR⁴;
each occurrence of R⁴ is independently selected from the group consisting of H, an optionally substituted C₁-C₁₀ alkyl, and an optionally substituted aryl-(C₁-C₄)alkyl;
each occurrence of t is independently an integer of 1-10; and
n is an integer of 1-20.

4. (canceled)

5. The method of claim 3, wherein the compound having the structure of Formula (XVII) (XII) is generated by reacting a compound having the structure of 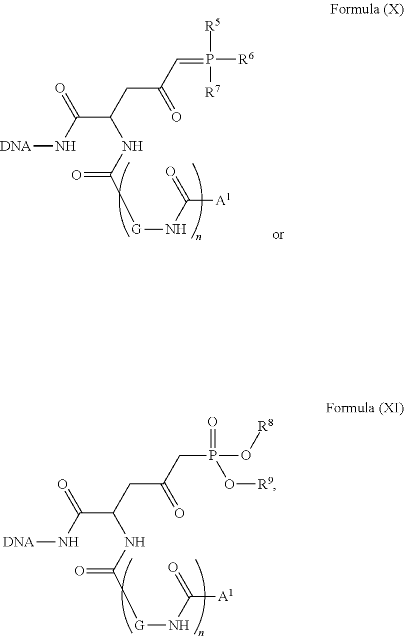
with compound having the structure of 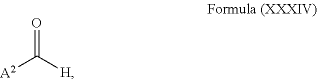
wherein DNA is a nucleic acid barcode;
each instance of G is independently of the formula 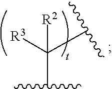
each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NR⁴;
each occurrence of R⁴ is independently selected from the group consisting of H, an optionally substituted C₁-C₁₀ alkyl, and an optionally substituted aryl-(C₁-C₄)alkyl;
each occurrence of R⁵, R, and R⁷ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R⁸ and R⁹ is independently selected from the group consisting of C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of t is independently an integer of 1-10; and
n is an integer of 1-20.

6. The method of claim 5,
a) wherein the compound having the structure of Formula (X) is generated by extending the peptidyl chain of a compound having the structure of 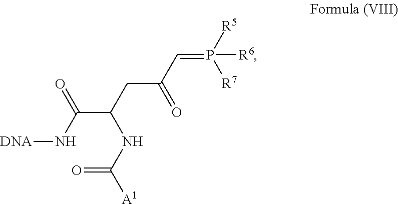
b) wherein the compound having the structure of Formula (XI) is generated by extending the peptidyl chain of a compound having the structure of 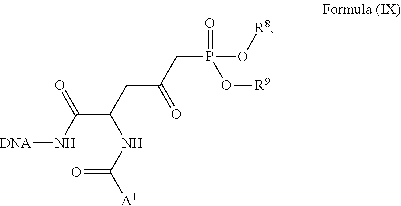
wherein DNA is a nucleic acid barcode;
A¹ is selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof, and
each occurrence of R⁵, R⁶, R⁷, R⁸, and R⁹ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

7. The method of claim 6,
a) wherein the compound having the structure of Formula (VIII) is generated by conjugating the deprotected amine of a compound having the structure of 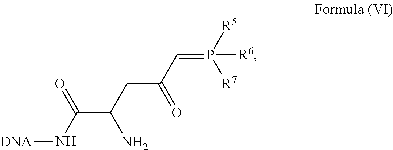
to a carboxylic acid of interest;
b) wherein the compound having the structure of Formula (IX) is generated by conjugating the deprotected amine of a compound having the structure of 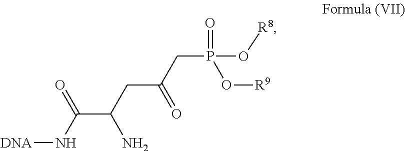
to a carboxylic acid of interest;
wherein DNA is a nucleic acid barcode; and
each occurrence of R⁵, R⁶, R⁷, R⁸, and R⁹ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof.

8. The method of claim 7,
a) wherein the compound having the structure of Formula (VI) is generated by removing the protecting group from a compound having the structure of 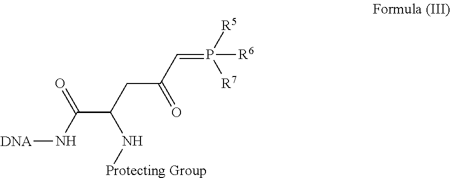
b) wherein the compound having the structure of Formula (VII) is generated by removing the protecting group from a compound having the structure of 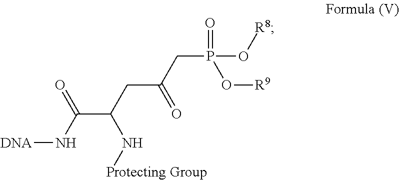
wherein DNA is a nucleic acid barcode; and
each occurrence of R⁵, R⁶, R⁷, R⁸, and R⁹ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof; and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

9. The method of claim 8,
a) wherein the compound having the structure of Formula (III) is generated by reacting a nucleic acid barcode with a compound having the structure of Formula (II) in the presence of a peptide coupling reagent, wherein Formula (II) is represented by 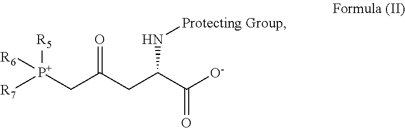
b) wherein the compound having the structure of Formula (V) is generated by reacting a nucleic acid barcode with a compound having the structure of Formula (IV) in the presence of a peptide coupling reagent, wherein Formula (IV) is represented by 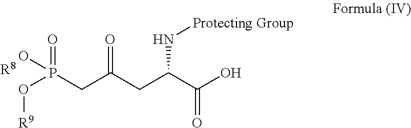
wherein each occurrence of R⁵, R⁶, R⁷, R⁸, and R⁹ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof, and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

10-14. (canceled)

15. A compound having a structure selected from the group consisting of: 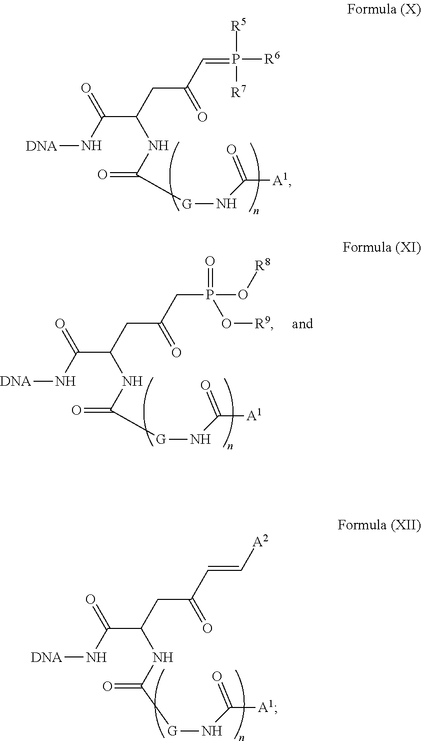
wherein each instance of G is independently of the formula 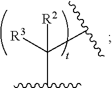
each occurrence of A¹ and A² is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of R² and R³ is independently selected from the group consisting of H, F, Cl, Br, I, N(R⁴)₂, OH, SH, an optionally substituted C₁-C₁₀ alkyl, an optionally substituted C₁-C₁₀ alkenyl, an optionally substituted C₁-C₁₀ alkynyl, an optionally substituted C₁-C₁₀ alkoxy, an optionally substituted C₁-C₁₀ alkylamino, an optionally substituted C₁-C₁₀ alkylthio, an optionally substituted C₃-C₁₀ cycloalkyl, an optionally substituted C₁-C₁₀ heteroalkyl, optionally substituted C₂-C₁₀ heterocyclyl, an optionally substituted aryl, an optionally substituted aryl-(C₁-C₄)alkyl, an optionally substituted heteroaryl, an optionally substituted aminoaryl, fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl;
Y is selected from the group consisting of OH, SH, and NR⁴;
each occurrence of R⁴ is independently selected from the group consisting of H, an optionally substituted C₁-C₁₀ alkyl, and an optionally substituted aryl-(C₁-C₄)alkyl;
each occurrence of R⁵, R⁶, R⁷, R⁸, and R⁹ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof;
each occurrence of t is independently an integer of 1-10; and
n is an integer of 1-20.

16. The compound of claim 15, wherein the compound has a structure selected from the group consisting of at least one structure as shown in FIGS. 3, 6-8, 14, 16, and 17.

17-22. (canceled)

23. An on-DNA phosphorane-ylide compound having a structure selected from the group consisting of: 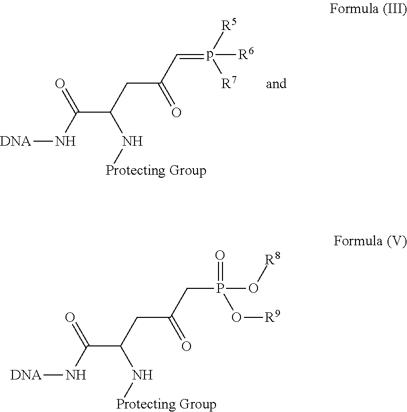
wherein each occurrence of R⁵, R⁶, R⁷, R⁸, and R⁹ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof, and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

24. The compound of claim 23, wherein the compound has a structure selected from the group consisting of at least one structure as shown in FIG. 1.

25. The compound of claim 23,
a) wherein the compound of Formula (III) is a compound of Formula (IIIa): 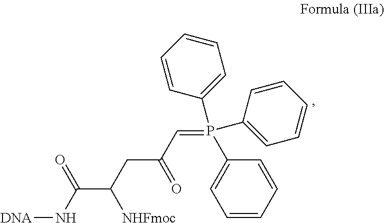
b) wherein the compound of Formula (V) is a compound of Formula (Va):

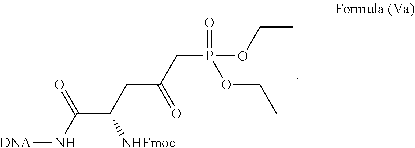

26-27. (canceled)

28. A method of preparing the on-DNA phosphorane ylide compound of claim 23 comprising:
a) obtaining a DNA barcode;
b) obtaining a phosphonium salt of Formula (II) or a β-keto phosphonate of Formula (IV): 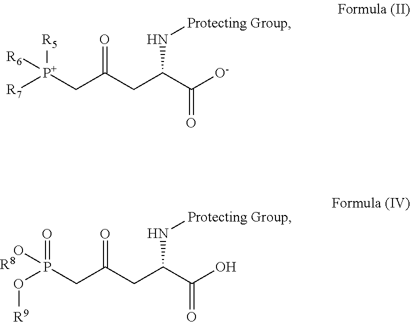
c) conjugating the phosphonium salt or a β-keto phosphonate of step (b) to an amine of the DNA barcode of step (a)
wherein each occurrence of R⁵, R⁶, R⁷, R⁸, and R⁹ is independently selected from the group consisting of H, halo, amino, thio, hydroxyl, C₁-C₁₀ alkyl, C₁-C₁₀ alkenyl, C₁-C₁₀ alkynyl, C₁-C₁₀ alkoxy, C₁-C₁₀ alkylamino, C₁-C₁₀ alkylthio, C₃-C₁₀ cycloalkyl, C₁-C₁₀ heteroalkyl, C₂-C₁₀ heterocyclyl, aryl, aryl-(C₁-C₄)alkyl, heteroaryl, and any combination thereof, and
the protecting group is selected from the group consisting of fluorenylmethyloxycarbonyl, tert-butyloxycarbonyl, benzyloxycarbonyl, acetyl, trifluoroacetyl, phthalimidyl, benzyl, triphenylmethyl, benzylidenyl, and tosyl.

29. (canceled)
